(19) United States
(12) Patent Application Publication
(10) Pub. No.: US 2006/0074225 A1
(43) Pub. Date: Apr. 6, 2006

(54) Monomeric immunoglobulin Fc domains

(75) Inventors: Aaron Keith Chamberlain, Pasadena, CA (US); John R. Desjarlais, Pasadena, CA (US)

Correspondence Address: DORSEY & WHITNEY LLP, 555 CALIFORNIA STREET, SUITE 1000, SUITE 1000, SAN FRANCISCO, CA, 94104, US

(73) Assignee: Xencor, Inc., Monrovia, CA (US)

(21) Appl. No.: 11/228,026

(22) Filed: Sep. 14, 2005

Related U.S. Application Data
(60) Provisional application No. 60/610,101, filed on Sep. 14, 2004.

(51) Int. Cl. C07K 16/18

(52) U.S. Cl. 530/387.1

(57) ABSTRACT

Design and production of immunoglobulin Fc domains including variants to stabilize their monomeric forms are provided.

This application claims benefit under 35 U.S.C. § 119(e) to U.S. Provisional Application No. 60/610,101, filed Sep. 14, 2004, which is incorporated herein by reference in its entirety.

### FIELD OF THE INVENTION

The invention relates to the design and production of stable monomeric immunoglobulin Fc domains.

### BACKGROUND OF THE INVENTION

Antibodies bind to specific antigens and consist of two heavy chains and two light chains covalently linked by a disulfide bonds (Janeway, et al. Immunobiology, 2001, 732, entirely incorporated by reference). Both the heavy and light chains contain variable regions, which bind the antigen, and constant regions. Upon protease cleavage, a dimer of the heavy chain constant regions, the Fc domain, is cleaved from the Fab domain. FIG. 1 illustrates a complete IgG antibody and identifies the sites of interactions with various proteins.

The variable region of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are six CDRs total, three each per heavy and light chain, designated V_{H} CDR1, V_{H} CDR2, V_{H} CD3, V_{L} CDR1, V_{L} CDR2, and V_{L} CDR3. The variable region outside of the CDRs is referred to as the framework (FR) region. Although not as diverse as the CDRs, sequence variability does occur in the FR region among different antibodies. Overall, this characteristic architecture of antibodies provides a stable scaffold (the FR region) upon which substantial antigen binding diversity (the CDRs) may be explored by the immune system to obtain specificity for a broad array of antigens. A number of high resolution structures are available for a variety of variable region fragments from different organisms, some unbound and some in complex with antigen. The sequence and structural features of antibody variable regions are well characterized (Morea et a., 1997, Biophys Chem 68:9-16; Morea et al., 2000, Methods 20:267-279, both entirely incorporated by reference), and the conserved features of antibodies have enabled the development of a wealth of antibody engineering techniques (Maynard et al., 2000, Annu Rev Biomed Eng 2:339-376, entirely incorporated by reference). For example, it is possible to graft the CDRs from one antibody, for example a murine antibody, onto the framework region of another antibody, for example a human antibody. This process, referred to in the art as “humanization”, enables generation of less immunogenic antibody therapeutics from nonhuman antibodies. Fragments comprising the variable region can exist in the absence of other regions of the antibody, including for example the antigen binding fragment (Fab) comprising V_{H}-Cγ1 and V_{L}-C_{L}, the variable fragment (Fv) comprising V_{H} and V_{L}, the single chain variable fragment (scFv) comprising V_{H} and V_{L} linked together in the same chain, as well as a variety of other variable region fragments (Little et al., 2000, Immunol Today 21:364-370, entirely incorporated by reference).

In humans, there are five isotypes, or classes, of heavy chains, delta (δ), gamma (γ), mu (μ), alpha (α) and epsilon (ε), giving rise to the IgD, IgG, IgM, IgA and IgE classes of antibodies. The IgA and IgG classes contain the subclasses, IgA1, IgA2, IgG1, IgG2, IgG3, and IgG4. The Fc regions of IgG, IgD and IgA dimerize through their Cγ3, Cδ3, and Cα3 domains, whereas the Fc regions of IgM and IgE dimerize through their Cμ4 and Cε4 domains.

The non-covalent interactions between the two IgG1 Fc domains are strong enough to maintain dimerization without the disulfide bonds (Ellerson, et al., 1976, J Immunol 116:510-517; Angal, et al. 1993, Mol Immunol 30:105-108; Ridgway, et al., 1996, Protein Eng 9:617-621; Atwell, et al., 1997, J Mol Biol 270:26-35; Dall'Acqua, et al., 1998, Biochemistry 37:9266-9273; DeLano, et al., 2000, Science 287:1279-1283; Schuurman, et al., 2001, Mol Immunol 38:1-8, all entirely incorporated by reference). Two Fc polypeptides joined covalently or non-covalently are referred to as an “Fc dimer”. Unfortunately, the literature contains many references to an “Fc monomer” that actually refer to two Fc polypeptides joined either covalently or non-covalently (Shan, et al., 1999, J Immunol 162:6589-6595; Wu, et al., 2001, Protein Eng 14:1025-1033; Kroez, et al., 2003, Biologicals 31:277-286, all entirely incorporated by reference). In those cases, the researchers studied the tendency of Fc dimers to form tetramers, hexamers and larger oligomers, which may occur. Measuring the molecular weight reveals the oligomerization state of the polypeptide. A monomeric Fc domain has approximately 225 amino acids and a molecular weight of approximately 25,000 Daltons, without a carbohydrate or any other attached molecules.

Previous research identified many residues that are important for Fc dimerization. The three dimensional structure of the IgG1 Fc domain demonstrates that the binding surface between two Fc monomers contains a central patch of hydrophobic residues surrounded largely by charged amino acids (DeLano et al., 2000, Science 287:1279-1283, entirely incorporated by reference). Alanine mutations in the central residues greatly affect the dimer stability, decreasing the free energy of unfolding by 2.1 to 2.5 kcal/mole (Dall'Acqua et al., 1998, Biochemistry 37:9266-9273, entirely incorporated by reference). The other mutations created in the Fc interface were designed to create Fc heterodimers. Ridgway et al. changed Thr366 to Tyr in one monomer and Tyr407 to Thr or Ala in another monomer and showed that mixtures of the two mutants result in 92% heterodimer formation (Ridgway et al., 1996, Protein Eng 9:617-621, U.S. Pat. No. 05,731,168, U.S. Pat. No. 05,807,706, U.S. Pat. No. 05,821,333, all entirely incorporated by reference). Atwell et al. have mutated Thr 366 on one monomer and selected for compensatory mutations in the other monomer using phage display at positions 366, 368 and 407 (Atwell et al., 1997, J Mol Biol 270:26-35, entirely incorporated by reference). They found heterodimers were more stable in one triple mutant, T366S/L368G/Y407V, and four other mutants in which at least one residue of 366, 368, and 407 was changed to Ala. All of these mutants were designed to form heterodimers.

Although the above experiments identified the residues in the Fc dimer interface, none of the created mutants have been shown to exist predominantly as folded monomers. Size exclusion chromatography under native solution conditions shows the mutants are dimers with no detectable monomeric polypeptides (Dall'Acqua et al., 1998, Biochemistry 37:9266-9273; Atwell et al., 1997, J Mol Biol 270:26-35, both entirely incorporated by reference). Weakening the dimerization is accomplished by mutating the residues in the interface, but unless the mutations make favorable interactions in the folded monomer, the disruption of the dimer will lead to an unfolded monomer. Mutations in the dimer interface that make stabilizing interactions within the folded monomer, however, will lead to an increase in the population of folded monomers. Therefore, one aspect of the present invention is to construct Fc mutations that stabilize the folded monomer.

The Fc region of an antibody interacts with a number of Fc receptors and ligands, imparting an array of important functional capabilities referred to as effector functions. For IgG, the Fc region comprises Ig domains Cγ2 and Cγ3 and the N-terminal hinge leading into Cγ2 (FIG. 1). An important family of Fc receptors for the IgG isotype are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290, all entirely incorporated by reference). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65, entirely incorporated by reference). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and γδ T cells.

Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290, all entirely incorporated by reference). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP). A number of structures have been solved of the extracellular domains of human FcγRs, including FcγRIIa (pdb accession code 1H9V, entirely incorporated by reference)(Sondermann et al., 2001, J Mol Biol 309:737-749, entirely incorporated by reference) (pdb accession code 1FCG, entirely incorporated by reference )(Maxwell et al., 1999, Nat Struct Biol 6:437-442, entirely incorporated by reference), FcγRIIb (pdb accession code 2FCB, entirely incorporated by reference)(Sondermann et al., 1999, Embo J 18:1095-1103, entirely incorporated by reference); and FcγRIIb (pdb accession code 1E4J, entirely incorporated by reference)(Sondermann et al., 2000, Nature 406:267-273, entirely incorporated by reference). All FcγRs bind the same region on Fc, at the N-terminal end of the Cγ2 domain and the preceding hinge, shown in FIG. 1. This interaction is well characterized structurally (Sondermann et al., 2001, J Mol Biol 309:737-749, entirely incorporated by reference), and several structures of the human Fc bound to the extracellular domain of human FcγRIIb have been solved (pdb accession code 1E4K, entirely incorporated by reference) (Sondermann et al., 2000, Nature 406:267-273, entirely incorporated by reference) (pdb accession codes 1IIS and 1IIX, entirely incorporated by reference)(Radaev et al., 2001, J Biol Chem 276:16469-16477, entirely incorporated by reference), as well as has the structure of the human IgE Fc/FcεRIα complex (pdb accession code 1F6A, entirely incorporated by reference) (Garman et al., 2000, Nature 406:259-266, entirely incorporated by reference).

An effector function of the Fc domain is the binding to the complement protein, C1q, to mediate complement dependent cytotoxicity (CDC). A site on Fc that is overlapping but separate from the FcγR binding site serves as the interface for the complement protein C1q (FIG. 1). C1q forms a complex with the serine proteases C1r and C1s to form the C1complex. C1q is capable of binding six antibodies, although the binding of two IgGs is sufficient to activate the complement cascade. Similar to Fc interaction with FcγRs, different IgG subclasses have different affinity for C1q, with IgG1 and IgG3 typically binding substantially better to the FcγRs than IgG2 and IgG4 (Jefferis et al., 2002, Immunol Lett 82:57-65, entirely incorporated by reference). There is currently no structure available for the Fc/C1q complex; however, mutagenesis studies have mapped the binding site on human IgG for C1q to a region involving residues D270, K322, K326, P329, and P331, and E333 (Idusogie et al., 2000, J Immunol 164:4178-4184; Idusogie et al., 2001, J Immunol 166:2571-2575, all entirely incorporated by reference).

A site on Fc between the Cγ2 and Cγ3 domains (FIG. 1) mediates interaction with the neonatal receptor FcRn, the binding of which recycles endocytosed antibody from the endosome back to the bloodstream (Raghavan et al., 1996, Annu Rev Cell Dev Biol 12:181-220; Ghetie et al., 2000, Annu Rev Immunol 18:739-766, all entirely incorporated by reference). This process, coupled with preclusion of kidney filtration due to the large size of the full-length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. Thus the fidelity of this region on Fc is important for both the clinical properties of antibodies and their purification. Available structures of the rat Fc/FcRn complex (Martin et al., 2001, Mol Cell 7:867-877, entirely incorporated by reference) and of the complexes of Fc with proteins A and G (Deisenhofer, 1981, Biochemistry 20:2361-2370; Sauer-Eriksson et al., 1995, Structure 3:265-278; Tashiro et al., 1995, Curr Opin Struct Biol 5:471-481, all entirely incorporated by reference) provide insight into the interaction of Fc with these proteins.

Although IgG is the generally the principal antibody isoform used for therapeutic applications, other isoforms have therapeutic potential. For example, a growing body of evidence suggests that interaction of IgA Fc with its Fc receptor FcαRI (CD89) elicits a plethora of effector functions (Egmond et al., 2001, Trends in Immunology, 22: 205-210, entirely incorporated by reference). IgA is the most prominent isotype of antibodies at mucosal surfaces, and the second most predominant isotype in human serum. A number of recent studies using bispecific antibody fragment constructs that simultaneously target a cancer antigen and FcαRI indicate that engagement of FcαRI can result in cell-mediated tumor cell killing (Stockmeyer et al., 2000, J. Immunol. 165: 5954-5961; Stockmeyer et al., 2001, J. Immunol. Methods 248: 103-111; Sundarapandiyan et al., 2001, J. Immunol. Methods 248: 113-123; dDechant et al., 2002, Blood 100: 4574-80, all entirely incorporated by reference). In addition, another study has shown that anti-FcγRI and FcαRI bispecific antibodies in combination provide synergistic anti-tumor efficacy, indicating that simultaneously targeting gamma and alpha Fc receptors may provide a means for enhancing the anti-cancer efficacy of antibodies and Fc fusions (van Egmond et al., 2001, Cancer Research 61: 4055-4060, entirely incorporated by reference). The structure of the extracellular domain of FcαRI has recently been solved (Ding et al., 2003, J. Biol. Chem. 278: 27966-27970, entirely incorporated by reference), as has the receptor in complex with IgA Fc (Herr et al., 2003, Nature 423: 614-620, entirely incorporated by reference), and the interface has been characterized with mutagenesis (Wines et al., 1999, J. Immunol., 162: 2146-2153; Wines et al., 2001, J. Immunol. 166: 1781-1789, both entirely incorporated by reference). FcαxRI binds to IgA Fc at a site between the Cγ2 and Cγ3 domains. Despite substantial structural homology between gamma and alpha Fc and FcRs, the IgA/FcαRI interaction is structurally distinct on Fc from the IgG/FcγR interaction.

The features of antibodies discussed above (i.e., specificity for target, ability to mediate immune effector mechanisms, and long half-life in serum) make antibodies powerful therapeutics. Monoclonal antibodies are used therapeutically for the treatment of a variety of conditions including cancer, inflammation, and cardiovascular disease. There are currently over ten antibody products on the market and hundreds in development.

Fc Fusion proteins are finding an expanding role in research and therapy (Chamow et al., 1996, Trends Biotechnol 14:52-60; Ashkenazi et al., 1997, Curr Opin Immunol 9:195-200, entirely incorporated by reference). An Fc fusion is a protein wherein one or more polypeptides are operably linked to Fc. An Fc fusion combines the Fc region of an antibody, and thus its favorable effector functions and pharmacokinetics, with the target-binding region of a receptor, ligand, or some other protein or protein domain. The role of the lafter is often to mediate target recognition, and thus it is functionally analogous to the antibody variable region. Variants of the present invention have utility in Fc fusions.

Despite such widespread use, antibodies and Fc fusions are not optimized for clinical use. A significant deficiency of antibodies and Fc fusions is their suboptimal anticancer potency. Another deficiency is the limited number of methods for their systemic delivery. This and other shortcomings of antibodies and Fc fusions are addressed by the present invention.

There are a number of possible mechanisms by which antibodies destroy tumor cells, including anti-proliferation via blockage of needed growth pathways, intracellular signaling leading to apoptosis, enhanced down regulation and/or turnover of receptors, CDC, ADCC, ADCP, and promotion of an adaptive immune response (Cragg et al., 1999, Curr Opin Immunol 11:541-547; Glennie et al., 2000, Immunol Today 21:403-410, both entirely incorporated by reference). Anti-tumor efficacy can be due to a combination of these mechanisms, and their relative importance in clinical therapy appears to be cancer dependent. Despite this arsenal of anti-tumor weapons, the potency of antibodies as anti-cancer agents is unsatisfactory, particularly given their high cost. Patient tumor response data show that monoclonal antibodies provide only a small improvement in therapeutic success over normal single-agent cytotoxic chemotherapeutics. For example, just half of all relapsed low-grade non-Hodgkin's lymphoma patients respond to the anti-CD20 antibody Rituxan® (rituximab) (Genentech/Biogenldec) (McLaughlin et al., 1998, J Clin Oncol 16:2825-2833, entirely incorporated by reference). Of 166 clinical patients, 6% showed a complete response and 42% showed a partial response, with median response duration of approximately 12 months. Trastuzumab (Herceptin®, Genentech), an anti-HER2/neu antibody for treatment of metastatic breast cancer, has lower efficacy. The overall response rate using trastuzumab for the 222 patients tested was only 15%, with 8 complete and 26 partial responses and a median response duration and survival of 9 to 13 months (Cobleigh et al., 1999, J Clin Oncol 17:2639-2648, entirely incorporated by reference). Currently for anticancer therapy, any small improvement in mortality rate defines success.

Protein therapeutics of smaller size have many favorable properties, including an increased ability to penetrate tumors. As is illustrated by single-chain antibody fragments (scFv's), smaller proteins more easily penetrate inside tumors (Yokota, et al., 1992, Cancer Res 52:3402-3408; Smith, 2001, Curr Opin Investig Drugs 2:1314-1319, both entirely incorporated by reference). The increase in tumor penetration may be seen as a more favorable tumor to blood ratio of protein. Additionally, smaller sized Fc fusions are more readily absorbed during pulmonary delivery (Bitonti, et al., 2004, Proc Natl Acad Sci USA 101:9763-9768, entirely incorporated by reference). The Fc fusion binds to FcRn in the lungs for transport to the circulation system. Although many other favorable properties are associated with smaller therapeutics, unfortunately their rate of renal clearance is increased. Therefore methods may need to be devised, e.g., increasing binding to FcRn, to increase the circulating half life of smaller therapeutics.

### SUMMARY OF THE INVENTION

In one aspect, the invention relates to the design and creation of stable, folded, monomeric Fc polypeptides. The invention provides the design, production methods, and therapeutic uses of monomeric Fc polypeptides. An Fc variant of the present invention preferably has at least one amino acid modification in the Fc region, and the resultant variant molecule has an increased content of folded, monomeric polypeptides and the polypeptides also have substantially reduced disulfide bonds. The variants of the present invention may be of any isotype, however, most preferred variants are human IgG.

In another aspect, the present invention is to provide Fc polypeptides from IgG, IgA, IgE, IgM and IgD isotypes with an increased content of folded, monomeric polypeptides in solution. The Fc polypeptides comprise at least one mutation in the interface between the Fc domains. In a preferred embodiment, the percentage of Fc polypeptides that are folded and monomeric will be greater than about fifty percent (50%).

It is another aspect of the present invention that the Fc variant of the present invention have at least one amino acid modification in the Fc region as compared to a wild type Fc region. The preferred modification(s) are selected 349, 351, 352, 353, 354, 356, 357, 364, 366, 368, 370, 392, 394, 395, 396, 397, 399, 405, 407 and 409; wherein the numbering is according to Kabat et al. The more preferred variants are 352, 353, 395, and 396; another preferred variant is 354. Preferably these variants are substituted with proline. In a further aspect, the Fc region is an IgG Fc region. In certain variations, the Fc variant can be greater than about a 50% monomer.

In another aspect, the modifications are not (a) a modification to alanine, (b) T366Y, (c) T366W, (d) Y407T, (e) T366S/L368A/Y407V, (f) T366S/L368V/Y407A, (g) L368A/Y407A, (h) T366S/L368A/Y407A, (i) T366S/L368G/Y407V, (j) F366Y/F405A, (k) T366W/F405W, (l) F405W/Y407Y, (m) T394W/Y407T, (n) T394S/Y407A, or (o) T366W/T394S.

In another aspect, the modifications include at least one of 349E, 349V, 351H, 351N, 352K, 353S, 354D, 356S, 357Q, 364A, 366E, 368Y, 368E, 370Q, 392E, 394N, 395N, 396T, 397Q, 399N, 405H, 405R, 407H, 407I, 409T and 409I.

In a further aspect, the Fc variants of the present invention with charged amino acid substitutions. The charged amino acid substitutions may be naturally occurring, synthetic or non-naturally occurring. However, naturally occurring substitutions are preferred. The most preferred substations include arginine, lysine, aspartate, glutamate, or histidine. The preferred variant positions for the charged amino acids are at least one of 368, 405, or 407, although at least two or three of these positions is preferred.

In an additional aspect, the Fc variant of the present invention have at least one deletion of at least one amino acid within residues 354 to 362 or 397 to 404, wherein the numbering is that of the EU index of Kabat et al.

### BRIEF DESCRIPTION OF THE DRAWINGS

FIG. 1. Antibody structure and function. Shown is a model of a full length human IgG1 antibody, modeled using a humanized Fab structure from pdb accession code 1CE1 (James et al., 1999, J Mol Biol 289:293-301, entirely incorporated by reference) and a human IgG1 Fc structure from pdb accession code 1DN2 (DeLano et al., 2000, Science 287:1279-1283, entirely incorporated by reference). The flexible hinge that links the Fab and Fc regions is not shown. IgG1 is a homodimer of heterodimers, made up of two light chains and two heavy chains. The Ig domains that comprise the antibody are labeled, and include V_{L} and C_{L} for the light chain, and V_{H}, Cgamma1 (Cγ1), Cgamma2 (Cγ2), and Cgamma3 (Cγ3) for the heavy chain. The Fc region is labeled. Binding sites for relevant proteins are labeled, including the antigen binding site in the variable region, and the binding sites for FcγRs, FcRn, C1q, and proteins A and G in the Fc region. The Cγ3/Cγ3 dimer interface is shown at the bottom.

FIG. 2. Equilibria governing the wild-type Fc domain and the Fc domains of the present invention. FIG. 2A shows the equilibria of the wild-type, dimeric Fc domain. The wild-type Fc domain exists predominantly as a folded dimer. If any folded monomeric species is present, it is undetectable, demonstrating that the equilibrium strongly favors the dimeric species under native conditions. Previous mutations in the Fc dimer interface have led to a decreased stability of the folded dimer relative to the unfolded monomeric state. FIG. 2B shows the equilibria that govern the designed Fc's of the present invention. The Fc mutants of the present invention were specifically designed to maintain the structure of the Fc in a monomeric state, while disrupting the dimeric structure. In these mutants, the folded monomer is now the predominant species under native conditions.

FIG. 3. Example calculations of the stability of each amino acid at positions in the IgG1 Cγ3 Fc domain monomer. The wild-type amino acids and positions are shown in the first two columns. The 10 most favorable substitutions (identity and energy) at the position are shown in the next 10 columns.

FIG. 4. Proline residues 352, 353, 395 and 396 and their influence on human IgG Cy3 domain structure.

FIG. 5. Serine 364 in the human IgG Cγ3 domain.

FIG. 6. Core residues 368, 405 and 407 and other residues 366, 370, and 409, which are both important in making interactions to stabilize the dimeric state.

FIG. 7. Example predictions of favorable double mutants in the monomeric IgG Fc. The positions, amino acids, energies and rank of each double mutant are shown. Predictions involving the wild-type amino acid in either position were deleted, leaving the list containing only double mutants and skips in rank.

FIG. 8. Energetically favorable triple variants at core positions 368, 405 and 407.

FIG. 9. Example energies of amino acid in various positions in the monomeric IgA1 Fc domain. Shown in the columns from right to left are the wild-type amino acid, the residue position number and the amino acids considered. The energy of each amino acid at the position is shown underneath the amino acid. More favorable amino acids at a given position have lower energy and appear toward the left side of the table. Numbering is according to Herr et al. 2003. Nature 423:614-620, entirely incorporated by reference.

FIG. 10. Example energies of amino acid in various positions in the IgE Fc domain. Shown in the columns from right to left are the wild-type amino acid, the residue position number and the amino acids considered. The energy of each amino acid at the position is shown underneath the amino acid. More favorable amino acids at a given position have lower energy and appear toward the left side of the table.

FIG. 11. Multimerization states of Fc fusion proteins. FIG. 11A shows an illustration of the aggregation of a fusion of the wild-type, dimeric Fc and its fusion partner, an oligomeric polypeptide. One molecule of the Fc fusion can bind to another Fc fusion using its Fc domain and can bind to at least one other Fc fusion using the partner domain. Since both ends of the fusion protein can bind another copy of the fusion protein, a multimer of indefinite length results. This aggregation interferes with the handling and function of Fc fusion proteins. FIG. 11B shows how the aggregation found in 11A is removed by fusing a monomeric Fc to the partner polypeptide. The fusion created with the monomeric Fc can only oligomerize using its partner domain creating a discrete multimer, which retains the oligomerization state of the fusion partner. Fc fusions can be created by fusing an Fc domain to a polypeptide with any oligomerization state. For illustrative purposes, a fusion partner with an oligomerization state of 3 is shown in the FIG. Thus, the fusion partner illustrated would often be referred to as a trimer.

FIG. 12. Example ACE™ scores describing the fitness of each amino acid at many sites in the IgG1 monomer structure. The residue positions and the wild-type amino acid are shown along the top and follow the numbering of Kabat et al. The permissiveness and precedence scores for each substituted amino acid are shown in the top and bottom block of numbers, respectively.

FIG. 13. Example ACE™ patch scores describing the fitness of each amino acid at site 368 in the monomeric IgG Fc. The patch amino acid is shown in one column. The columns to the right of the patch amino acid demonstrate the 18 sites that are most important in determining the environment around the patch site, 368. The relative strengths of each site in determining the environment are listed as fractional numbers below the residue numbers of each site and range between 0.4 and 0.02 in this example. Leu, the wild-type is shown to be the most favored amino acid. Amino acids with lower patch scores (left column) are listed toward the bottom of the table. The representative sequence chosen for each possible patch amino acid is that sequence that yielded the highest patch score. The names of these sequences are listed to the right of the patch scores.

FIG. 14. Example ACE™ patch scores describing the fitness of amino acid pairs at sites 405 and 407 in the monomeric IgG Fc. The table contents are similar to those shown in FIG. 13. Here, two positions are considered to be patch amino acids. Patches of any size can be specified by the user of the ACE™ programs. In this example, double mutations are suggested. The double mutants with the highest fitness for the monomeric IgG1 Fc structure are listed toward the top of the figure.

FIG. 15. Example ACE™ patch scores describing the fitness of amino acid pairs at sites 351 and 409 in the monomeric IgG Fc. The table contents are similar to those shown in FIG. 13. Here, two positions are considered to be patch amino acids. Patches of any size can be used. In this example, double mutations are suggested. The amino acids specified do not need to reside close to each other in the structure or sequences as is illustrated with these patch residues, 351 and 409. The double mutants with the highest fitness for the monomeric IgG1 Fc structure are listed toward the top of the figure.

FIG. 16. Some numbering conventions used herein. Exemplary human IgG, IgA, IgM, IgE, and IgD sequences are shown with the EU index of Kabat et al., the OU index of Kabat et al., the numbering of Herr et al. and the numbering of Garman et al. (Kabat, et al., 1991, Sequences and Proteins of Immunological Interest, United States Public Health Service, National Institutes of Health, Bethesda; Herr et al. 2003. Nature 423:614-620; Garman et al. 2000. Nature 406:259-266, all entirely incorporated by reference).

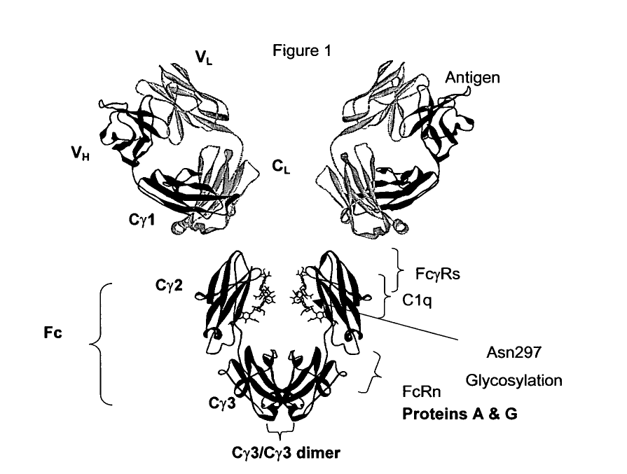

### DESCRIPTION OF THE INVENTION

In order that the invention may be more completely understood, several definitions are set forth below. Such definitions are meant to encompass grammatical equivalents. By “ADCC” or “antibody dependent cell-mediated cytotoxicity” as used herein is meant the cell-mediated reaction wherein nonspecific cytotoxic cells that express FcRs recognize bound antibody on a target cell and subsequently cause lysis of the target cell. By “ADCP” or antibody dependent cell-mediated phagocytosis as used herein is meant the cell-mediated reaction wherein nonspecific cytotoxic cells that express FcRs recognize bound antibody on a target cell and subsequently cause phagocytosis of the target cell. By “amino acid modification” herein is meant an amino acid substitution, insertion, and/or deletion in a polypeptide sequence. The preferred amino acid modification is a substitution. By “antibody” herein is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes respectively. The term “antibody” is meant to include full-length antibodies and antibody fragments, and may refer to a natural antibody from any organism, an engineered antibody, or an antibody generated recombinantly for experimental, therapeutic, or other purposes. By “amino acid” and “amino acid identity” as used herein is meant one of the 20 naturally occurring amino acids or any non-natural analogues that may be present at a specific, defined position. By “effector function” as used herein is meant a biochemical event that results from the interaction of an antibody Fc region with an Fc receptor or ligand. Effector functions include but are not limited to ADCC, ADCP, and CDC. By “effector cell” as used herein is meant a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and γδ T cells, and may be from any organism including but not limited to humans, mice, rats, rabbits, and monkeys. By “library” herein is meant a set of Fc polypeptides in any form, including but not limited to a list of nucleic acid or amino acid sequences, a list of nucleic acid or amino acid substitutions at variable positions, a physical library comprising nucleic acids that encode the library sequences, or a physical library comprising the Fc polypeptide proteins, either in purified or unpurified form. By “Fc” or “Fc region” as used herein is meant the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgG, Fc comprises immunoglobulin domains CH2 and CH3, also referred to as Cgamma2 and Cgamma3 (Cγ2 and Cγ3) and the hinge between Cgamma1 (Cγ1) and Cγ2. For IgA, Fc comprises immunoglobulin domains CH2 and CH3, also referred to as Calpha2 and Calpha3 (Cα2 and Cα3) and the hinge between Calpha1 (Cα1) and Cα2. Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an antibody, antibody fragment, or Fc fusion. By “Fc fusion” as used herein is meant a protein wherein one or more polypeptides is operably linked to Fc. Fc fusion is herein meant to be synonymous with the terms “immunoadhesin”, “Ig fusion”, “Ig chimera”, and “receptor globulin” (sometimes with dashes) as used in the prior art (Chamow et al., 1996, Trends Biotechnol 14:52-60; Ashkenazi et al., 1997, Curr Opin Immunol 9:195-200, entirely incorporated by reference). An Fc fusion combines the Fc region of an immunoglobulin with the target-binding region of a receptor, an adhesion molecule, a ligand, an enzyme, or some other protein or protein domain. The role of the non-Fc part of an Fc fusion is to mediate target binding, and thus it is functionally analogous to the variable regions of an antibody. By “Fc gamma receptor” or “FcγR” as used herein is meant any member of the family of proteins that bind the IgG antibody Fc region and are substantially encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIlb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et a., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes. By “Fc ligand” as used herein is meant a molecule, preferably a polypeptide, from any organism that binds to the Fc region of an antibody to form an Fc-ligand complex. Fc ligands include but are not limited to FcγRs, FcαRs, FcεRs, FcRn, C1q, C3, Fc receptor homologs (FcRH) including but not limited to FcRH1-6, FcRY (examples of FcRHs are described by Davis, RS et al., 2002, Immunological Reviews, 190: 123-136, entirely incorporated by reference), mannan binding lectin, mannose receptor, staphylococcal protein A, streptococcal protein G, and viral FcγR. Fc ligands may include undiscovered molecules that bind Fc. By “Fc polypeptide” as used herein is meant a polypeptide that comprises Fc. An Fc polypeptide may be an antibody, Fc fusion, or a protein or protein domain that comprises Fc. An Fc polypeptide may be naturally occurring, or may be an Fc polypeptide variant of a parent Fc polypeptide. By “full length antibody” herein is meant the structure that constitutes the natural biological form of an antibody, including variable and constant regions. For example, in most mammals, including humans and mice, the full length antibody of the IgG isotype is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In some mammals, for example in camels and llamas, IgG antibodies may consist of only two heavy chains, each heavy chain comprising a variable domain attached to the Fc region. By “IgG” as used herein is meant a polypeptide belonging to the isotype of antibodies that are substantially encoded by a recognized immunoglobulin gamma gene. In humans this isotype comprises IgG1, IgG2, IgG3, and IgG4. In mice this isotype comprises IgG1, IgG2a, IgG2b, and IgG3. By “immunoglobulin (Ig)” as used herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains. By “immunoglobulin (Ig) domain” as used herein is meant a region of an immunoglobulin that exists as a distinct structural entity as ascertained by one skilled in the art of protein structure. Ig domains typically have a characteristic β-sandwich folding topology. The known Ig domains in the IgG isotype of antibodies are V_{H}, Cγ1, Cγ2, Cγ3, V_{L}, and C_{L}. By “parent polypeptide” as used herein is meant an unmodified polypeptide that is subsequently modified to generate a variant. Said parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “Parent Fc polypeptide” as used herein is meant an unmodified Fc polypeptide that is modified to generate a variant, and by “parent antibody” as used herein is meant an unmodified antibody that is modified to generate a variant antibody. By “position” as used herein is meant a location in the sequence of a protein. Positions may be numbered sequentially, or according to an established format, for example the EU index as in Kabat. For example, position 297 is a position in the human antibody IgG1. By “residue” as used herein is meant a position in a protein and its associated amino acid identity. For example, asparagine 297 (also referred to as N297 or Asn297) is a residue in the human antibody IgG1. By “target antigen” as used herein is meant the molecule that is bound specifically by the variable region of a given antibody. A target antigen may be a protein, carbohydrate, lipid, or other chemical compound. By “target cell” as used herein is meant a cell that expresses a target antigen. By “variable region” as used herein is meant the region of an immunoglobulin that comprises one or more Ig domains substantially encoded by any of the Vκ, Vλ, and/or V_{H} genes that make up the kappa, lambda, and heavy chain immunoglobulin genetic loci respectively. By “variant polypeptide” as used herein is meant a polypeptide sequence that differs from that of a parent polypeptide sequence by virtue of at least one amino acid modification. A variant polypeptide may refer to the polypeptide itself, a composition comprising the polypeptide, or the amino sequence that encodes it. Preferably, the variant polypeptide has at least one amino acid modification compared to the parent polypeptide, e.g., from about one to about ten amino acid modifications, and preferably from about one to about five amino acid modifications compared to the parent. The variant polypeptide sequence herein will preferably possess at least about 80% homology with a parent polypeptide sequence, and most preferably at least about 90% homology, more preferably at least about 95% homology. Accordingly, by “Fc variant” as used herein is meant an Fc sequence that differs from that of a parent Fc sequence by virtue of at least one amino acid modification. An Fc variant may only encompass an Fc region, or may exist in the context of an antibody, Fc fusion, or other polypeptide that is substantially encoded by Fc. Fc variant may refer to the Fc polypeptide itself, compositions comprising the Fc variant, or the amino acid sequence that encodes it.

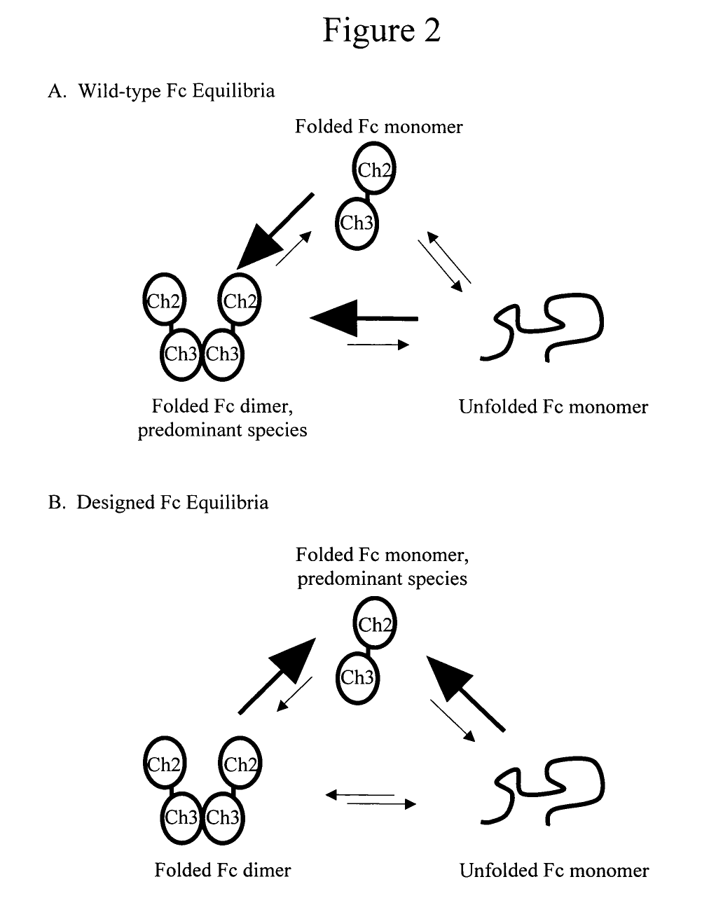

The invention discloses Fc domains that better retain their structure as a monomer and have an increased content of folded monomers. The wild-type Fc and previously constructed mutants of Fc exist predominantly as a dimer under native solution conditions (FIG. 2a) (Ellerson, et al., 1976, J Immunol 116:510-517; Angal, et al. 1993, Mol Immunol 30:105-108; Ridgway, et al., 1996, Protein Eng 9:617-621; Atwell, et al., 1997, J Mol Biol 270:26-35; Dall'Acqua, et al., 1998, Biochemistry 37:9266-9273; DeLano, et al., 2000, Science 287:1279-1283; Schuurman, et al., 2001, Mol Immunol 38:1-8, all entirely incorporated by reference). The Fc mutants in the present invention have a shift in their equilibrium to favor the folded, monomeric Fc domain (FIG. 2B). The Fc interface region is mutated to make favorable interactions in folded Fc monomer while also disrupting the dimer interface. Almost any mutation in the interface will lead to a decrease in dimer stability. The difficulty in designing a monomeric Fc domain is to retain monomer stability as the mutations disrupt the dimer.

PDA® technology, including ACE™ algorithms, was used in part to design the Fc variants of the present invention. PDA® algorithms use a search strategy and energy potential to assess the compatibility of a polypeptide sequence in a structure. ACE™ algorithms use a template structure and a multiple sequence alignment to assess the effect of substituting one or more amino acids into a protein structure. See, U.S. Pat. Nos. 6,188,965; 6,269,312; 6,403,312; 6,708,120; 6,792,356; 6,801,861; 6,804,611; and 6,864,359; U.S. Ser. Nos. 09/877,695; 10/071,859; 09/812,034; 10/888,748; 09/782,004; 09/927,790; 10/101,499; 10/218,102; 10/666,307; 10/666,311; 11/008,647; and 11/149,943; all hereby entirely incorporated by reference. PDA® algorithms use as input protein structures such as those available from the Protein Data Bank, PDB (Research Collaboratory for Structural Bioinformatics, RCSB). PDB structures of Fc domains, polypeptides comprising Fc domains and fragments of Fc domains include the following PDB codes 1ADQ, 1DN2, 1E4K, 1FC1, 1FC2, 1FCC, 1FP5, 1FRT, 1G84, 1H3T, 1H3U, 1H3V, 1H3W, 1H3X, 1H3Y, 1HZH, 1I1A, 1I1C, 1IGT, 1IGY, 1IIS, 1IIX, 1K6X, 1O0V, 1OQO, 1OQX, 1OW0, and 1T89, all entirely incorporated by reference. ACE™ algorithms use as input a protein structure and a multiple sequence alignment. Sequences and multiple sequence alignments for different proteins and protein families are available at the National Center for Biotechnology Information (NCBI). Alignments may be constructed with a variety of techniques including, BLAST and PSI-BLAST (Altschul, S. F., Madden, T. L., Schäffer, A. A., Zhang, J., Zhang, Z., Miller, W. & Lipman, D. J. (1997) Nucleic Acids Res. 25:3389-3402 and Combinatorial Extension of optimal path, C E, Shindyalov and Bourne (1998) Protein Engineering 11(9) 739-747, all entirely incorporated by reference).

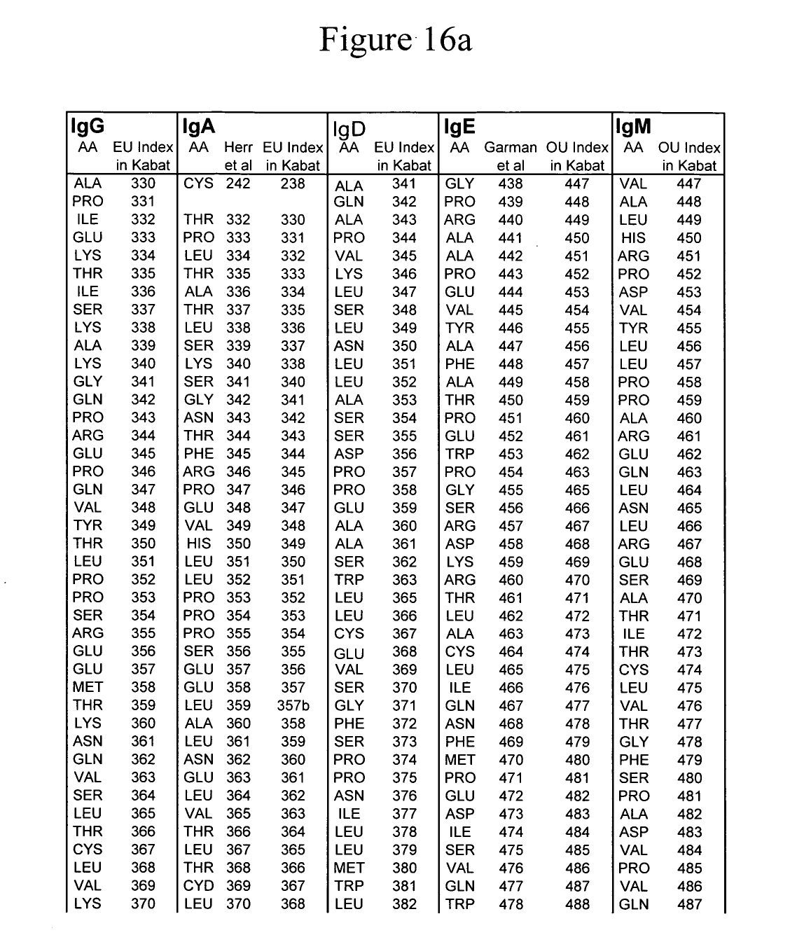

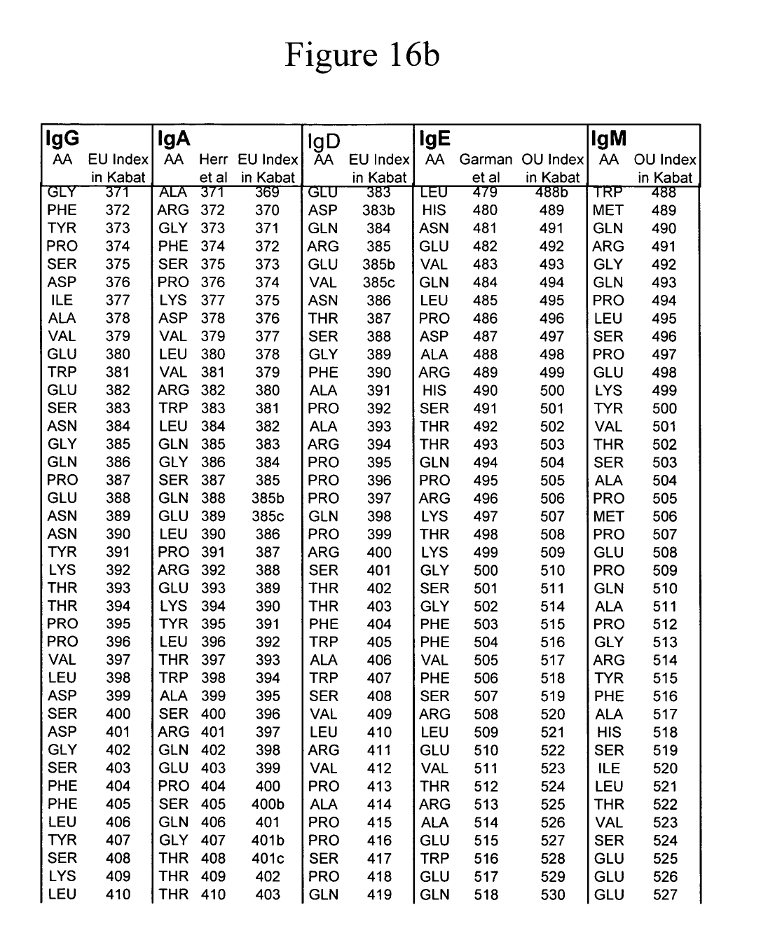

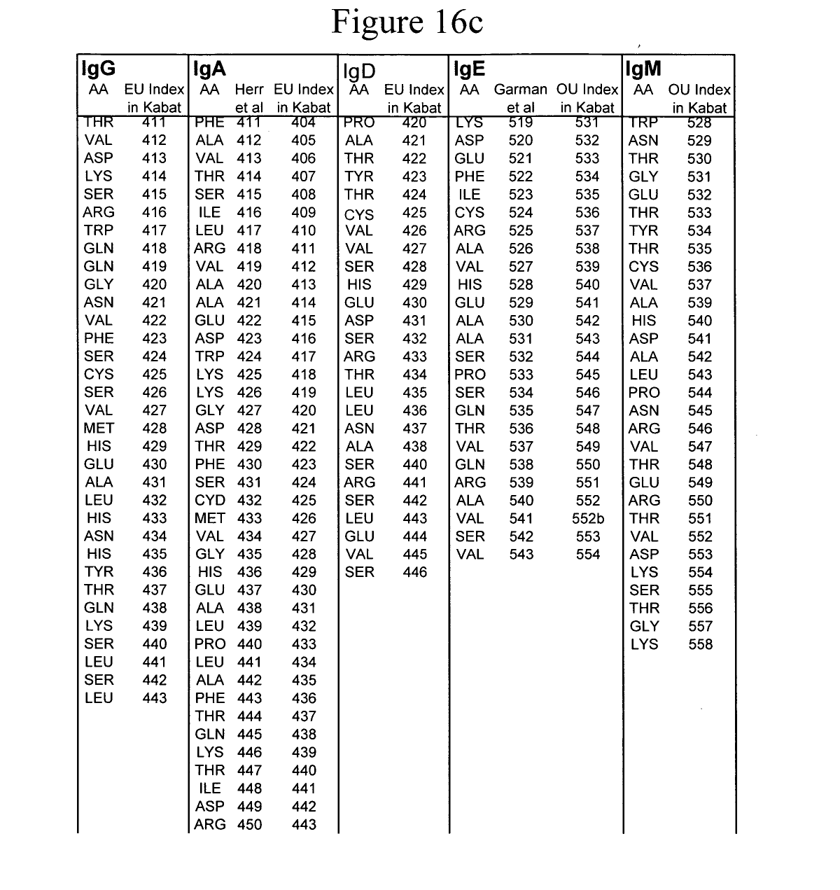

The designs of the mutants in the present invention are derived from various computational predictions, which employ PDA® and ACE™ technology as well as more traditional sequence and structure alignments. Using the EU numbering system of Kabat et al. (Kabat, et al., 1991, Sequences and Proteins of Immunological Interest, United States Public Health Service, National Institutes of Health, Bethesda, entirely incorporated by reference), the mutant positions in the Fc domain of IgG that increase the level of stable monomer are taken from, but not limited to the following positions: 261, 349, 351, 352, 353, 354, 356, 364, 366, 368, 370, 392, 394, 395, 396, 397, 399, 401,403, 405, 407, and 409. (Exemplary sequences of human IgG, IgA, IgE, IgD, and IgM with the some numbering conventions used herein are listed in FIG. 16). Wild type Fc regions are known in the art. Wild-type Fc regions include, for example, those disclosed in U.S. patent application Ser. Nos. 10/379,392, 10/672,280,10/822,231, 11/124,620, and PCT/US2005/023328, each of which is incorporated herein by reference in its entirety.

The two heavy chains in the dimer are often held together by disulfide bonds. These disulfide bonds need to be reduced or eliminated in order to create monomer Fc species, if the Cys residues are present in the protein comprising the Fc domain. One method of reducing the disulfide bond is by the addition of chemical reducing agents such as reduced S-adenocyl methionine (SAM), beta-mercaptoethonal (also know as 2-mercaptoethanol), dithiothreitol (DTT), or other reagents. The cysteine residues may also be substituted for another residue. Commonly used alternative residues include alanine and serine, although many amino acids may be used to forbid the disulfide bond, including amino acids not considered one of the twenty commonly found in proteins. Synthetic amino acids are well known in the art and include, for example WO 05/074,650. The cysteine residues may also be deleted from the protein, being part of deleted region of the protein. The deleted region may be any length greater than or equal to 1 residue, although deletions of 1 to 3 or 5 residues are preferred. An example of cysteine residue to be reduced or eliminated to favor the formation of monomeric Fc domains, includes Cys in the hinge region of human IgG sequences.

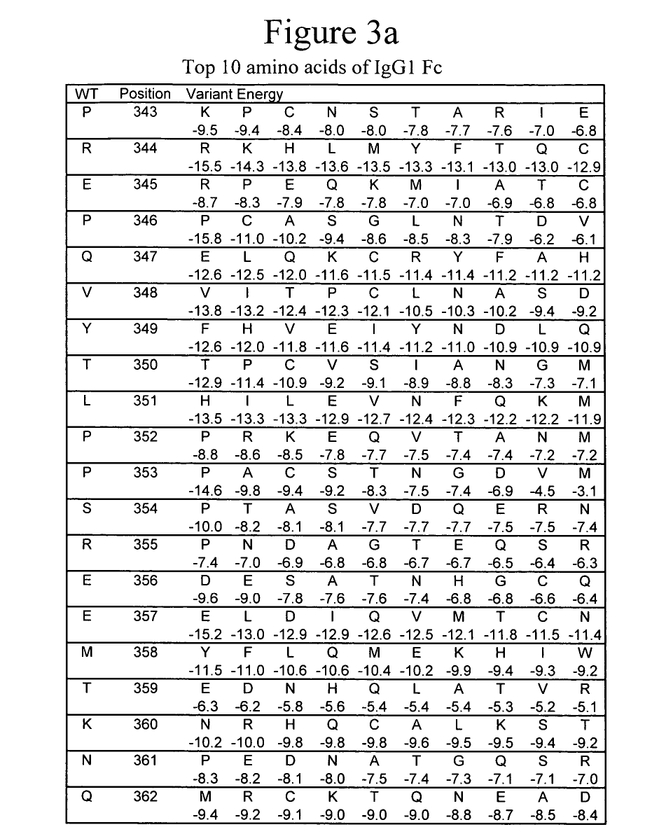

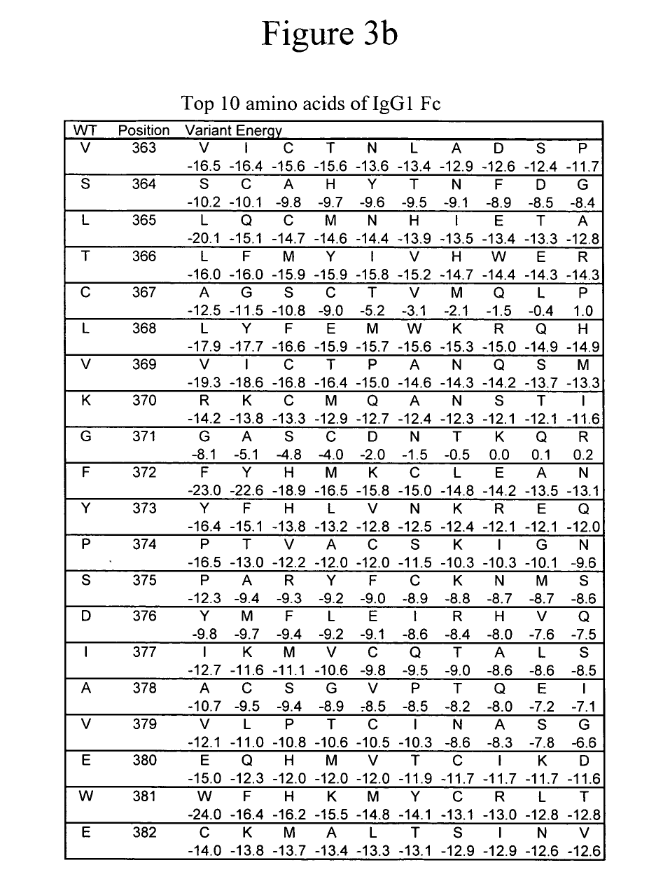

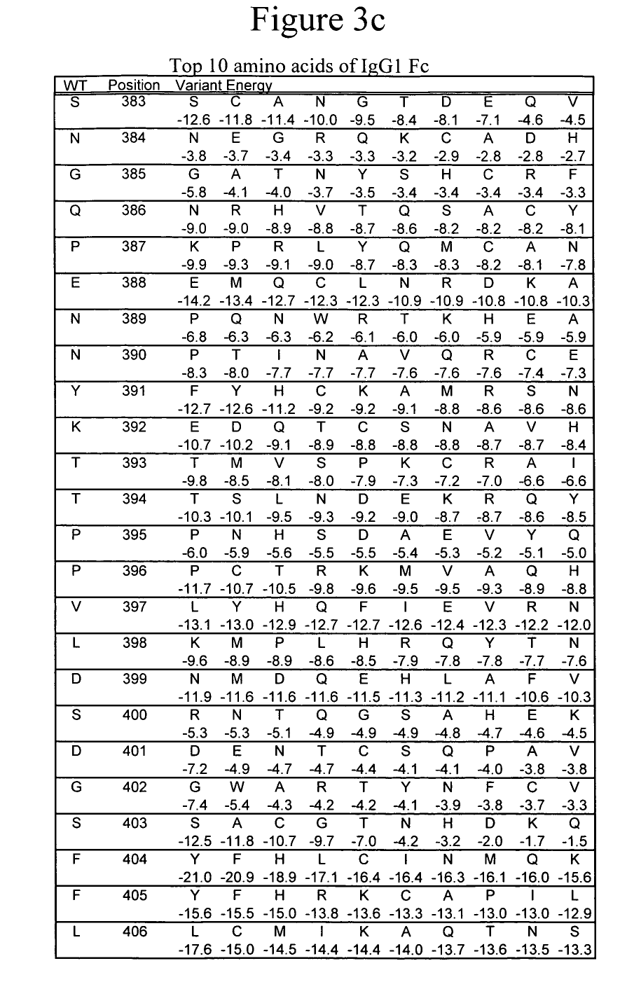

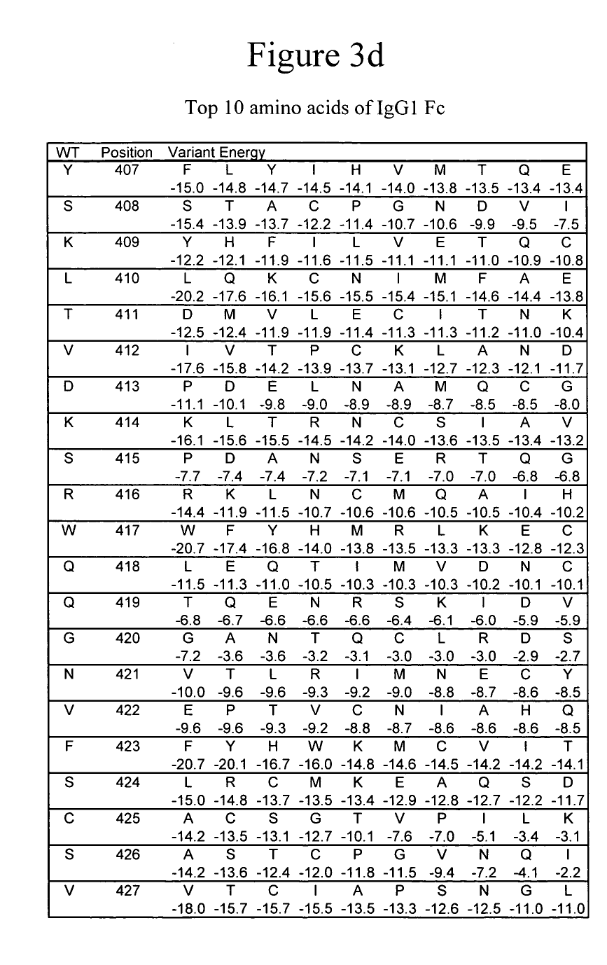

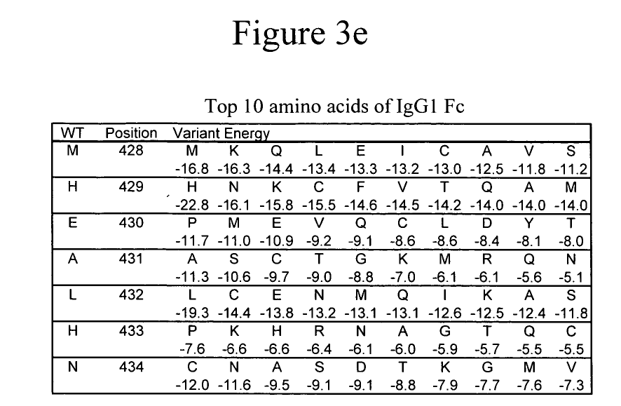

Point mutations that are predicted to increase the monomer content of IgG Fc include, but are not limited to, the following variants as shown by PDA® technologies (FIG. 3): Y349H, Y349V, Y349E, L351H, L351l, L351E, L351N, P352R, P352K, P352E, P352Q, P353A, P353S, P353N, S354P, S354T, S354D, E356D, E356S, E356A, E357L, E357Q, S364A, S364H, S364N, T366L, T366H, T366K, T366E, L368Y, L368E, L368K, L368R, L368Q, K370R, K370M, K370Q, K370A, K370N, K392E, K392E, K392Q, K392T, T394S, T394L, T394N, T394D, T394K, P395N, P395H, P395S, P395D, P396T, P396R, P396K, P396V, V397Y, V397H, V397Q, V397E, D399N, D399M, D399Q, D399H, D401E, D401N, D401T, S403A, S403N, F405Y, F405H, F405R, F405K, F405D, F405P, Y407L, Y407H, Y407T, Y407Q, K409Y, K409H, K4091, K409E and K409T.

These variants and other variants at these positions that are substitutions to charged residues are especially preferred because of their large disruption of the dimer interface from electrostatic effects. The charge on one monomer repels the like charge on the other monomer. Charge/charge interactions, or monopole/monopole interactions, create large disruptions, because the energy of their interaction decreases with the square of the distance between the charges as shown by Coulomb's law. Other interactions in proteins decrease with higher order powers of the distance between the two interacting centers. For example, dipole/dipole interactions decrease with increasing distance to the fourth power, and Van der Waals interactions, also known as London dispersion forces, decrease with increasing distance to the sixth power. These power dependences of the energy/distance relationships means that electrostatic interactions contribute to the stability of proteins over a much longer distance than Van der Waals interactions or dipole/dipole interactions such as hydrogen bonding. Therefore, two like charges, for example two positive charges, placed on opposite sides of the monomer/monomer interface can repel each other even if the atoms are not in direct contact, ie the atoms centers are not within the sum of their Van der Waals radii. Therefore, preferred substitutions for creating a monomeric Fc domain use charged amino acids including Arg, Lys, Asp, Glu, and His.

Charged amino acid in particular, but also polar amino acids more generally speaking, also make favorable interactions with water or solute molecules if they reside in exposed positions in a protein. Charged solute molecules, including salts and many buffer reagents, are common in in vitro and in vivo solutions containing proteins and help to stabilize proteins generally. Proteins have a strong preference for charged and polar amino acid on their exposed positions as oppose to their buried positions, demonstrating the added stability of exposed charged residues. In the process of making a monomeric Fc domain, a normally buried region, such as IgG positions 368, 405 and 407, become exposed to solvent and other solute molecules, suggesting that polar amino acids, preferably charged amino acids, should be used at those positions. Because placement of two or more like charges near each other in a protein can also disrupt the structure of the protein, particularly preferred variants do not include those with three like charges in positions 368, 405, and 407.

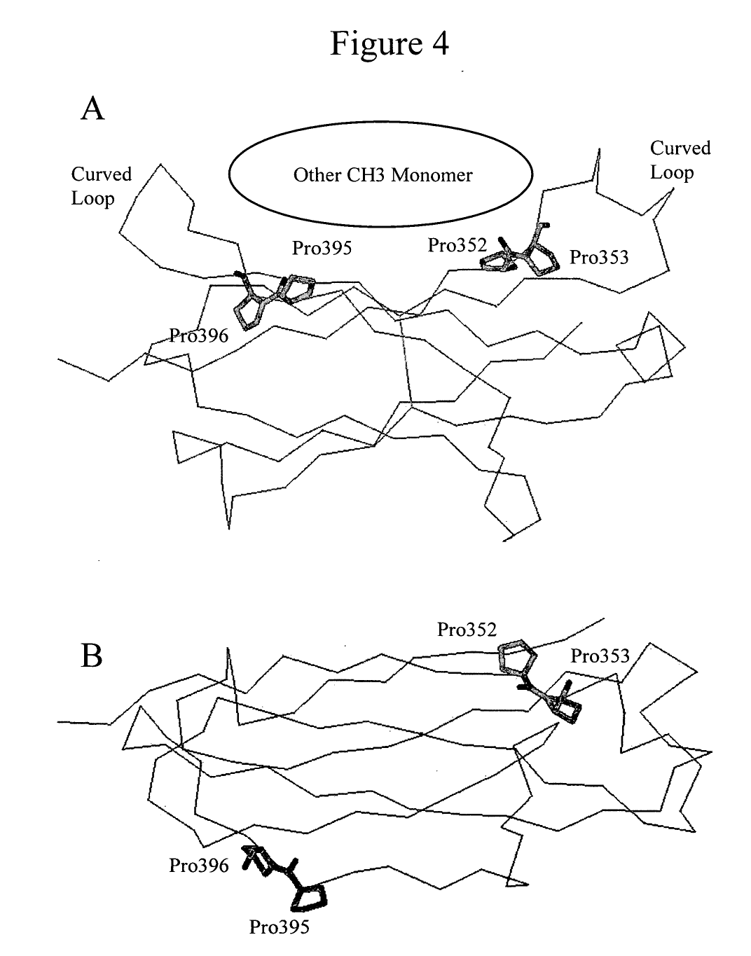

A particularly unique feature that helps hold together the Fc domain dimers is the curvature of the beta sheet structure and two loops that comprise the monomer/monomer interface. The two loops residing in this region curve drastically away from the remainder of their Fc monomer and make extensive contacts between the monomers (FIG. 4). In human IgG1, these loops comprise residues 354-362 and 397-404 using the numbering of the EU index of Kabat et al. Four proline residues play important roles in stabilizing the curvature in these loops, prolines 352, 353, 395 and 396 in the human IgG1. Two of these proline residues are located prior to each of the two curved loops. Proline residues are incompatible with beta sheet structures because their phi/psi angles are very limited and not favorable for beta sheets. The two prolines before each loop forbid the continuation of the beta strands and restrict the phi/psi angles to those that stabilize the curved backbone. To stabilize the monomeric forms of the CH3 domain, therefore, these prolines should be mutated to allow the loops to relax to a more favorable position after the removal of the adjacent monomer.

Substitution of these prolines for another residue should be done with a residue that is compatible with the monomer structure and reflects the newly exposed environment of the prolines in the monomer. As shown by the output of the PDA® design algorithms (FIG. 3), the variants P352R, P352K, P352E, and P352Q are energetically favorable at position 352. These substitutions favor the long, polar or charged amino acids that are good at making favorable electrostatic and hydrogen bonding interactions with water or solvent molecules. The algorithms demonstrate that positively charged residues are the most favorable substitutions with P352R and P352K having the lowest energy of any substitution using the 20 commonly found amino acids. At position 353, the small amino acids Ala, Cys, Ser, and Thr are favored reflecting the steric constraints at this position. Cys is generally unfavorable as a substituting amino acid because of its ability to form disulfide bonds (unwanted here) or to oxidize. At position 395, Asn, His, Ser and Asp are the favored substitutions, again demonstrating that polar residues generally are favored at this position in the monomer. These amino acids also fit in the position sterically, and received better (lower) energies than some other polar residues, such as Lys and Arg, which are in the lower half of energetically favorable residues, and not shown in FIG. 3. At position 396, Cys, Thr, Arg, and Lys are favored, with Cys being less favorable as explained above. This position, as did position 352, favors the positively charged amino acids, Arg and Lys.

The curved loops, positions 354-362 and 397-404 in following the EU numbering of Kabat et al., may also benefit from deletion some of their positions. These long, curved loops will loose contacts in a dimer to monomer transition. Their excessive length for the monomer can destabilize the folded monomer relative to an unfolded monomer. This decrease in folded monomer stability occurs because the entropy of the unfolded state grows more quickly than the entropy of the folded state as the loop length increases. Therefore, if a longer loop does not make compensatory energetic contacts, i.e. enthalpic contacts, then the longer loop destabilizes the folded protein. This phenomenon is explained by the basic polymer theories developed largely by Flory (Principles of Polymer Chemistry, P J Flory, 1953 Cornell University Press. 1st Edition, entirely incorporated by reference) Deletions of 1, 2, 3, 4, 5 or more residues from these loops will help reduce this unfavorable entropic effect. Deletion of too many residues from these loops, i.e. more than about 7 residues, will destabilize the folded species more than the unfolded species, as the strands before and after each loop will become sterically hindered.

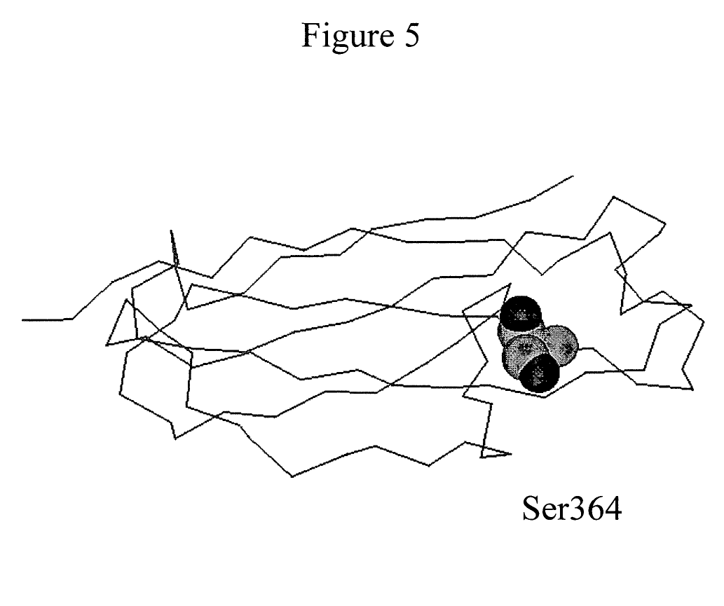

Position 364 is a key residue in the formation of the dimer interface and a key residue to help disrupt this interface in the stabilization of the monomeric CH3 domain. In human IgG1, this position contains a Ser residue (FIG. 5), which interacts with Y349 and T350 on the adjacent monomer in the dimeric form. Because of serine's small size, variants at this position to almost any other amino acid will increase the size of the side chain and “over-pack” the dimer interface, resulting in a destabilized dimer. Variants at this position to Thr comprise a conservative substitution that can over-pack the interface by the addition of a methyl group. The substitution to Thr is also favorable energetically (FIG. 3a). Proline is another favorable substitution at this location as judged by energetic considerations (FIG. 3a). Ala, Gly and Cys are the only amino acid of the 20 naturally occurring amino acids that will not over-pack position 354.

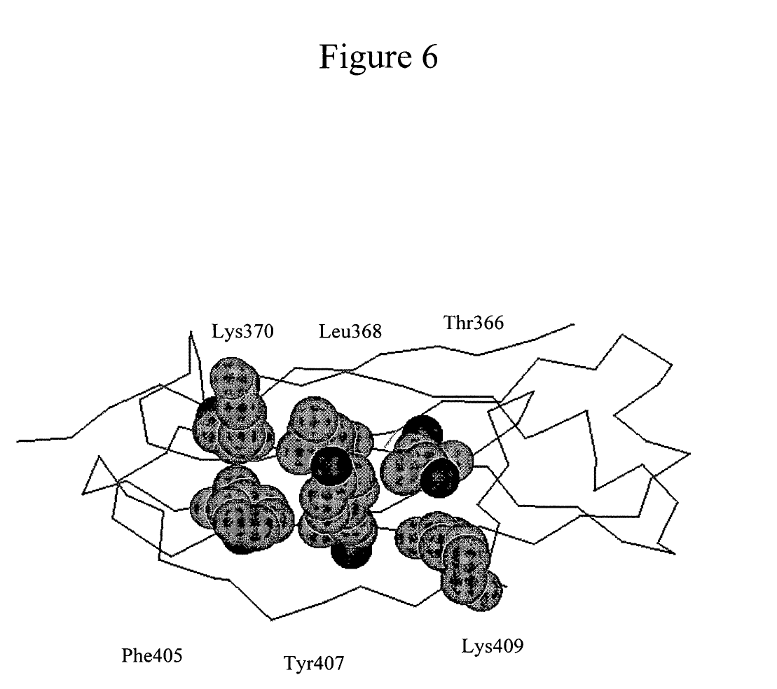

Positions 368, 405, and 407 are all hydrophobic amino acids (Leu, Phe, and Tyr) in human IgG1 and are the core residues in holding together the dimer interface. FIG. 6 shows these core residues and residues 366, 370, and 409 in a monomer of the PDB structure 1DN2 of human IgG1. The energetic calculations (FIG. 3B-D) suggest that point mutations to hydrophobic residues are favorable. Tyr and Phe are favorable point mutations at position 368, Phe is a favorable substitution at position 405, and Phe and Leu are favorable substitutions at position 407. These substitutions may be favorable as point mutations, but such conservative point mutations are unlikely to disrupt the monomer/monomer interface enough to create an isolated monomer. Combinations of these variants, variants to less conserved amino acids, and combinations of less conserved amino acid are preferred.

The polar substitutions are better substitutions at these positions (368, 405, 407) for stabilizing the isolated monomer. Polar residues that will disrupt the hydrophobic interactions comprising this interface include Gln, Glu, Asn, Asp, Lys, Arg, His, Ser, Thr, and Gly. The longer amino acids are also likely to create steric clashes if the monomers come in close proximity. The long, polar amino acids comprise Gln, Glu, Asn, Asp, Lys, Arg, and His. Although all polar substitutions will be beneficial in disrupting the dimer interface, certain polar residues are preferred at these three positions because of their interactions with neighboring residues. As shown in FIG. 3B-D, favorable polar residues at position 368 include Glu, Lys, Arg, Gin, and His. Favorable polar residues at position 405 include His, Arg, and Lys. Favorable polar residues at position 407 include His, Thr, Gln, and Glu.

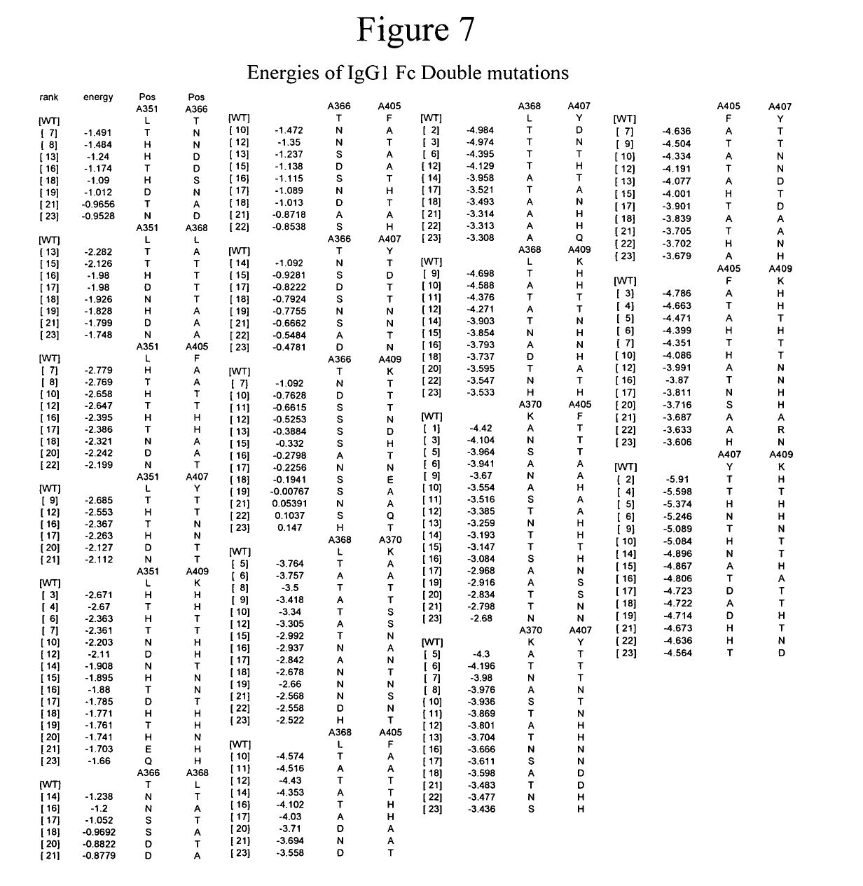

Double substitution variants comprising a variant at position 368, 405, or 407 in IgG, or their analogous residues in other isotypes, have a greater capacity to disrupt the monomer/monomer interface and destabilize the dimeric Fc. FIG. 7 shows favorable, double-substitution variants at positions 368, 405, 407 and others. For example, in human IgG1, the double variant L368T/Y407D is predicted to be the most favorable double variant at positions 368 and 407. In addition, L351T/T366N is preferred at positions 351 and 366. Other higher order mutations of IgG Fc include, but are not limited to, L368R/F405Q, L368R/Y407D, L368T/Y407D, L368E/F405K, L368K/Y407E, L368R/F405Q, L368R/F405Q, K370D/D399K, L368T/Y407D, L368R/F405Q/L351S, and L351S/K392S/T394R/V397E/F405T/Y407T. The most preferred combinations of mutations do not necessarily comprise the most preferred single mutations, because a mutation at one position in the Fc can interact with mutations at one or more different positions.

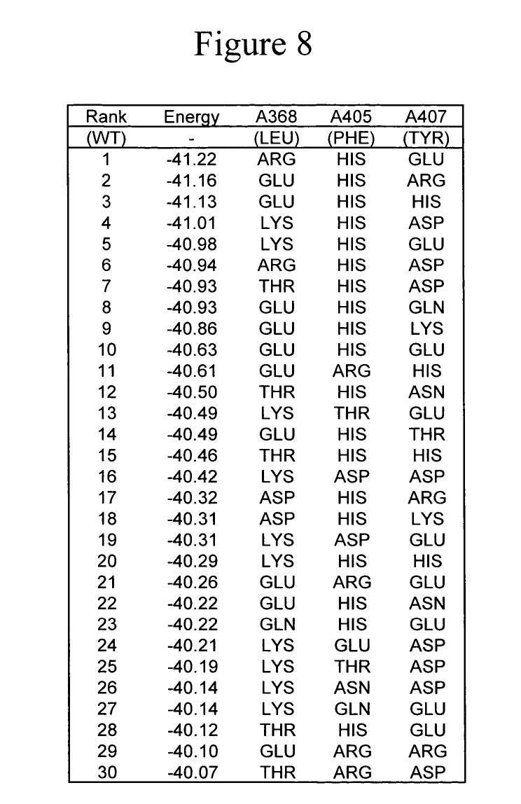

Combinations of substitutions of positions 368, 405 and 407 are particularly powerful in disrupting the interface. Triple variants that replace the wild-type residues with polar residues will disrupt the hydrophobic interactions at the core of the interface. The substituting amino acids should be chosen such that the three new amino acids are compatible with each other and with the surrounding amino acids. FIG. 8 shows the 30 preferred triple substitutions to polar amino acids at these positions as determined by PDA® technologies. The energies of the triple variants, in general, are fairly similar, demonstrating that the three sites do not have an absolute requirement for a particular set of three polar amino acids. Upon inspection, however, it can be seen that position 405 is often predicted to have a His residue. If fact, the top 10 ranking combinations all contained His at position 405. Changing the human IgG1 Phe residue to a His residue retains the ring nature of the side chain. In contrast, position 407 also has a ring-containing side chain in the human IgG1 sequence, a Tyr, but the PDA® algorithms do not predict that retention of the ring chain is favorable. At this position, combinations with long, straight, polar residues are found.

The modification of these three residues to polar residues in a monomer brings about favorable interactions with water. The polar residues can make hydrogen bonding interactions with water and the removal of exposed hydrophobic interactions allows and increase in water entropy, which is largely responsible for the hydrophobic effect, the separation of water and oily hydrophobic substances (Proteins: Structure and Molecular Properties, T E Creighton, 2^{nd} Edition, 1992, W. H. Freeman Publishers, entirely incorporated by reference).

Other isotypes or sub-classes of antibodies can also be mutated in an analogous manner to the Cγ3 domain of IgG1 antibodies. The dimerization domain and the numbering of the residues will differ between isotypes, but in all cases the dimerization domains are homologous to IgG1. Mutations in the Ch3 domains of IgG, IgA and IgD isotypes can create Fc monomers, whereas mutations in the Ch4 domains of IgE and IgM isotypes can create Fc monomers.

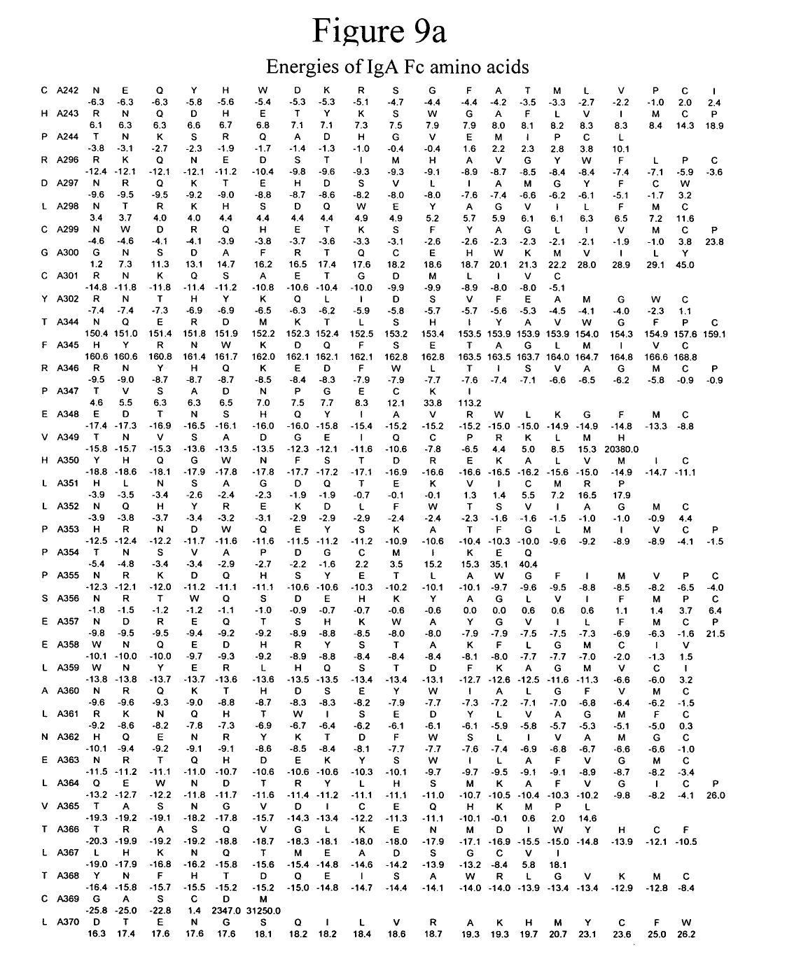

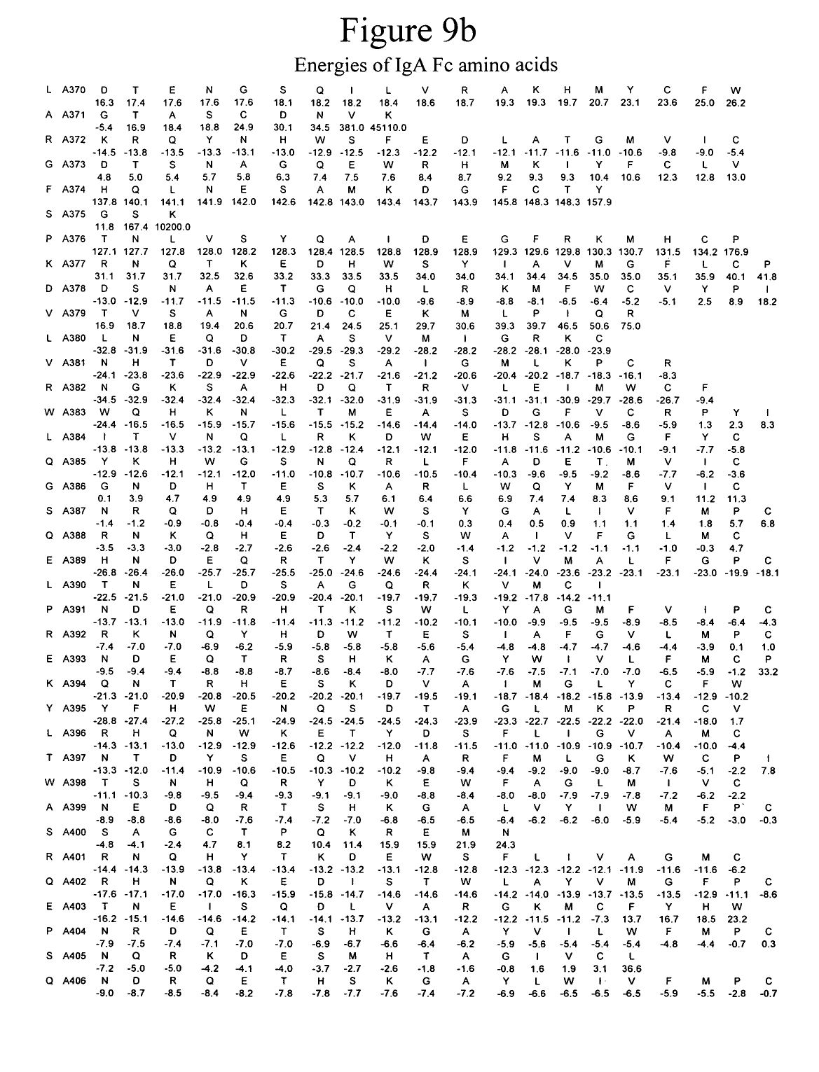

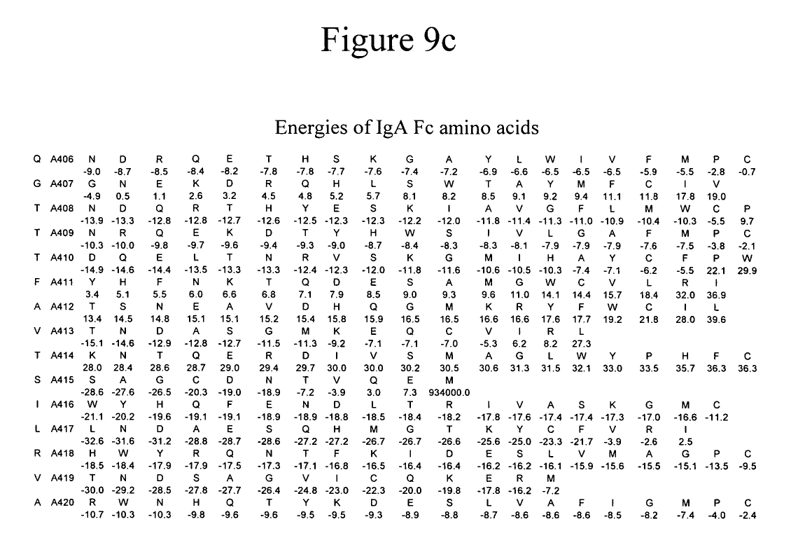

In IgA, using the numbering scheme in Herr et al. (Herr et al. 2003. Nature 423:614-620, entirely incorporated by reference), point mutations that are predicted to increase the amount of folded monomer occur in, but are not limited to, the following positions: 242, 298, 299, 301, 350, 352, 353, 354, 355, 357, 358, 366, 368, 370, 372, 393, 394, 395, 396, 397, 398, 399, 400, 401,402, 403, 404, 412, 413, 414, 416 and 418. (Exemplary sequences of human IgG, IgA, IgE, IgD, and IgM with the some numbering conventions used herein are listed in FIG. 16.) An IgA monomer is of particular interest, because binding of IgA to an IgA receptor does not require the participation of two IgA polypeptides as shown in the structure of the IgA/IgARI complex, PDB code 1OW0.pdb, entirely incorporated by reference; (Herr et al. 2003. Nature 423:614-620, entirely incorporated by reference). Therefore, monomers of IgA should still be able to bind to IgA receptors and undergo the effectors functions that require this interaction (for example, ADCC). Removal of the cysteine residue at position 242 is beneficial for forbidding the formation of the inter-chain disulfide bond. The cysteine at positions 299 and 301 can also be removed to forbid unwanted disulfide bonds. Most preferred mutations occur in the following positions: 352, 368, 370, 396, 398, 401, 412, 414 and 416. Preferred variants at these positions (FIG. 9) include L352N, L352H, L352R, T368Y, T368N, T368F, L370D, L370T, L370E, L396R, L396H, L396Q, W398T, W398S, W398N, W398H, R401N, R401Q, R401H, A412T, A412N, A412E, T414K, T414N, I1416Y, I416H, l416Q, and I416E.

In IgA, using the EU index numbering scheme of Kabat et al. (Kabat, et al., 1991, Sequences and Proteins of Immunological Interest, United States Public Health Service, National Institutes of Health, Bethesda, incorporated by reference), point mutations that are predicted to increase the amount of folded monomer occur in, but are not limited to, the following positions: 238, 294, 295, 297, 349, 351, 352, 353, 354, 356, 357, 364, 366, 368, 370, 389, 390, 391, 392, 393, 394, 395, 396, 397, 398, 399, 400, 405, 406, 407, 409 and 411. The cysteine mutants to forbid the formation of disulfide bonds occur at positions: 238, 295 and 297. Most preferred mutations occur in the following positions: 351, 366, 368, 392, 394, 397, 405, 407 and 409.

Although an atomic structure of IgD Fc is not available, by analogy to the simulations on IgG and IgA Fc domains and using the sequence alignment and EU numbering scheme of Kabat et al. (Kabat, et al., 1991, Sequences and Proteins of Immunological Interest, United States Public Health Service, National Institutes of Health, Bethesda, entirely incorporated by reference), point mutants that are predicted to increase the amount of folded monomer occur in, but are not limited to, the following positions: 238, 294, 295, 297, 349, 351, 352, 353, 354, 356, 357, 364, 366, 368, 370, 389, 390, 391, 392, 393, 394, 395, 396, 397, 398, 399, 400, 405, 406, 407, 409 and 411. A cysteine mutation to forbid the formation of disulfide bonds occurs in the hinge region. Most preferred mutations occur in the following positions: 351, 366, 368, 392, 394, 397, 405, 407 and 409.

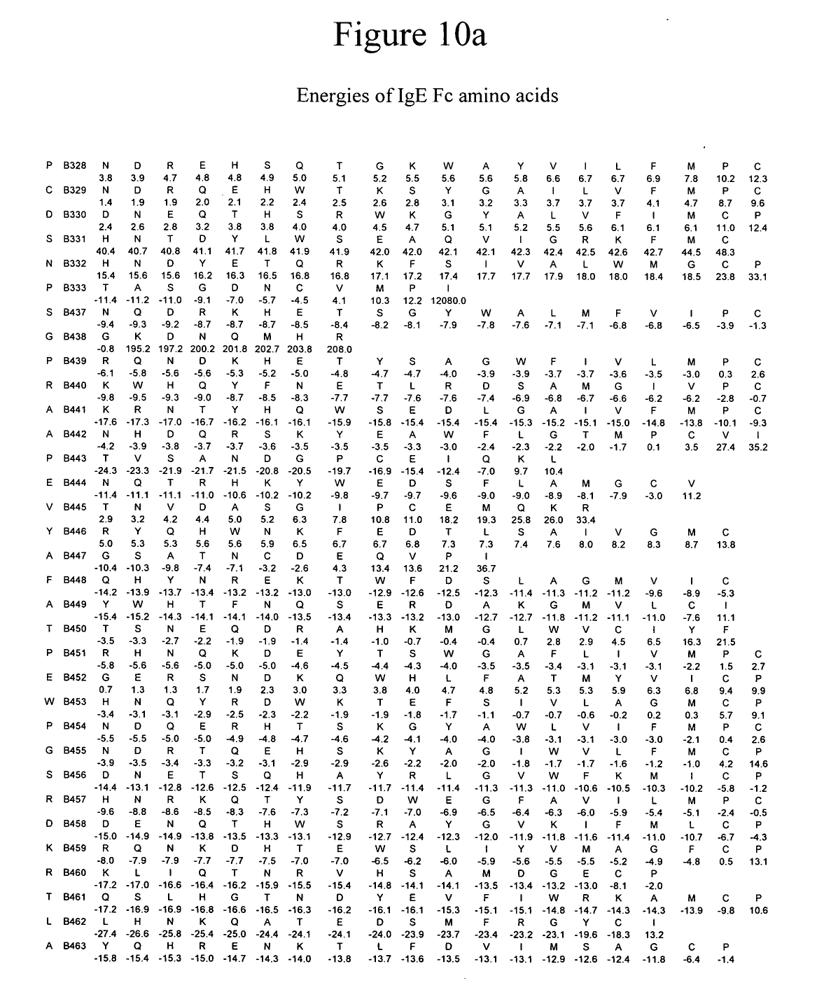

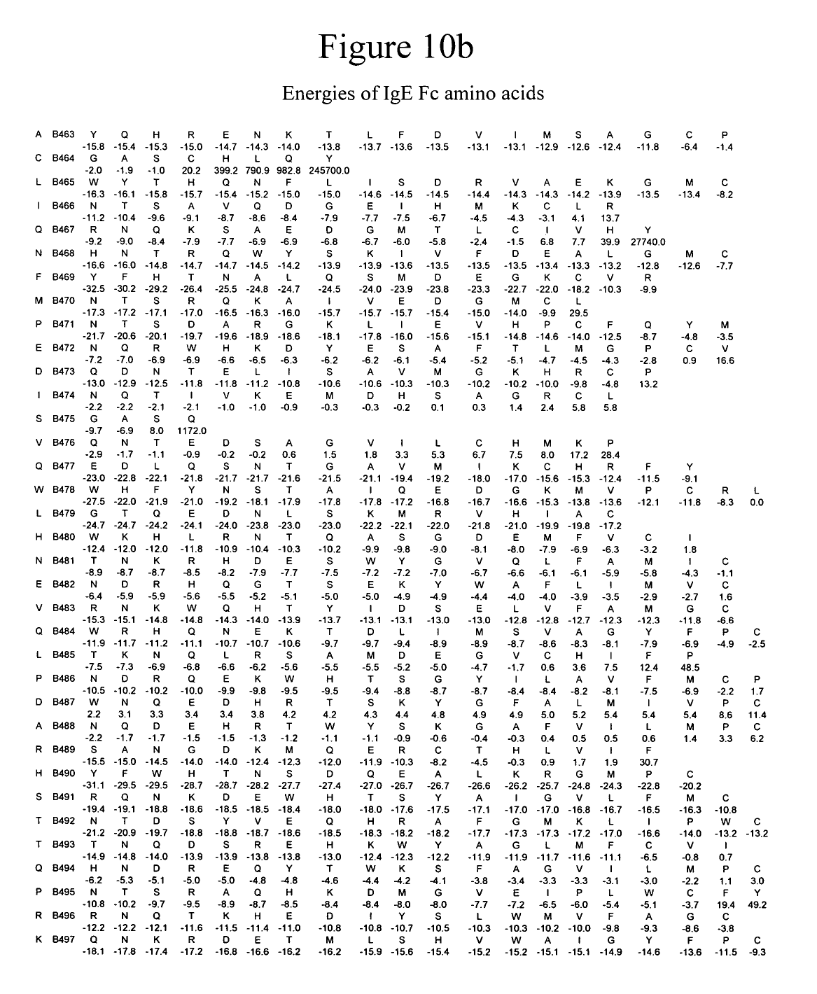

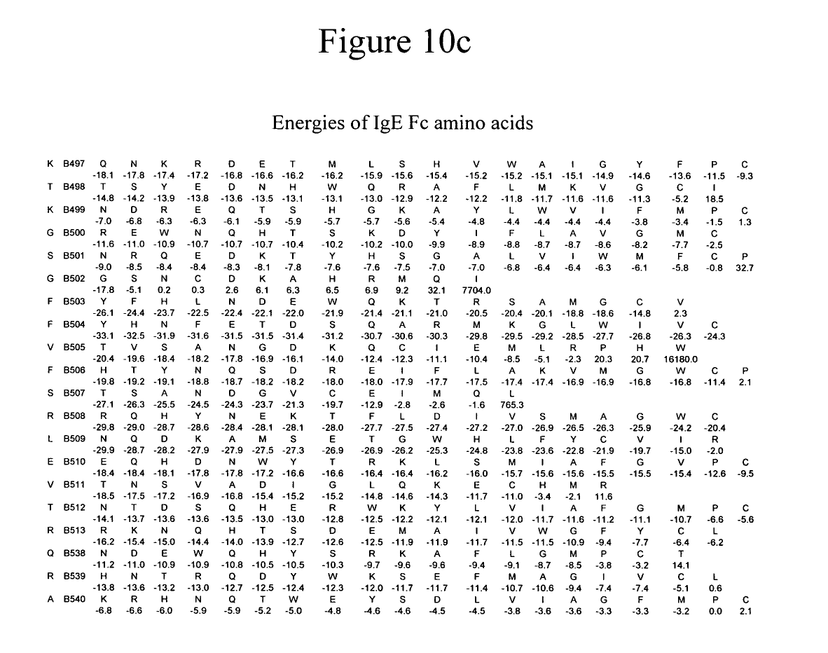

In IgE, using the numbering scheme of Garman et al., (Garman et al. 2000. Nature 406:259-266, entirely incorporated by reference), point mutations that are predicted to increase the amount of folded monomer occur in, but are not limited to, the following positions: 328, 329, 331, 332, 442, 443, 444, 445, 446, 447, 448, 449, 450, 451, 452, 453, 455, 456, 461, 462, 463, 464, 465, 467, 468, 489, 491, 492, 493, 494, 496, 498, 499, 500, 502, 504, 505, 506, 507, 508, 510 and 539. (Exemplary sequences of human IgG, IgA, IgE, IgD, and IgM with the some numbering conventions used herein are listed in FIG. 16). Removal of the cysteine residues at positions 261 and 329 may be used to forbid the formation of disulfide bonds. Most preferred variants occur in the following positions: 446, 448, 463, 465, 504, 506 and 508. FIG. 10 shows the preferred substitutions at these positions.

In IgE, using the OU index numbering scheme of Kabat et al., point mutations that are predicted to increase the amount of folded monomer occur in, but are not limited to, the following positions: 337, 338, 340, 341, 451, 452, 453, 454, 455, 456, 457, 458, 459, 460, 461, 462, 465, 466, 471, 472, 473, 474, 475, 477, 478, 499, 501, 502, 503, 504, 506, 508, 509, 510, 514, 516, 517, 518, 519, 520, 522 and 551. The removal of cysteine residues is beneficial for forbidding the formation of the inter-chain disulfide bond. Most preferred mutations occur in the following positions: 455, 457, 473, 475, 516, 518 and 520.

Although an atomic resolution structure of IgM Fc is not available, by analogy to the simulations on IgE Fc domains and using the sequence alignment and OU numbering scheme of Kabat et al. (Kabat, et al., 1991, Sequences and Proteins of Immunological Interest, United States Public Health Service, National Institutes of Health, Bethesda, entirely incorporated by reference), point mutations that are predicted to increase the amount of folded monomer occur in, but are not limited to, the following positions: 337, 338, 340, 341, 451, 452, 453, 454, 455, 456, 457, 458, 459, 460, 461, 462, 465, 466, 471, 472, 473, 474, 475, 477, 478, 499, 501, 502, 503, 504, 506, 508, 509, 510, 514, 516, 517, 518, 519, 520, 522 and 551. The removal of the cysteine residues is beneficial for forbidding the formation of the inter-chain disulfide bond. Most preferred mutations occur in the following positions: 455, 457, 473, 475, 516, 518 and 520.

The variant amino acids in the present invention may be entirely incorporated into antibodies, Fc fusions, or other proteins comprising at least a portion of the Fc domain. The variants may be entirely incorporated into proteins derived from any organism, including humans, mice, rats, rodents, primates, monkeys, camels, alpacas, llamas, camelids with humans, rodents and primates being preferred and humans being most preferred.

Many of the designed, monomeric Fc domains are unlikely to retain the function of antibody-dependent cytotoxicity (ADCC), because the antibody receptors bind both copies of the variable region in the full-length antibody. This lack of FcR binding may be useful in antibody or Fc fusion proteins in cases where receptor stimulation is not desired. IgA Fc's are an exception as their receptors bind at the Cα2/Cα3 interface within one monomer. In addition, the neo-natal Fc receptor (FcRn) only binds one Fc monomer suggesting that the Fc monomers of the present invention will largely retain FcRn binding.

Another aspect of the present invention is that alterations in the effector functions of the Fc domain may occur during monomerization. For example, the IgG Cγ2 domain may have more flexibility to move relative to the Cγ3 domain in the monomer structure. Additional mutations can be made to the Fc in order to maintain the Fc effector functions. In one example, the Fc binding to the FcRn can be adjusted by mutations in the FcRn/Fc interface or in Cγ2/Cγ3 domain interface. These mutations include, but are not limited to, positions: 248, 249, 250, 251, 252, 253, 254, 255, 256, 257, 258, 284, 285, 286, 287, 288, 306, 307, 308, 309, 310, 311, 312, 313, 314, 315, 382, 385, 387, 426, 427, 428, 429, 430, 431, 432, 433, 434, 435, 436, 437, 246, 247, 248, 249, 250, 251, 252, 253, 254, 310, 311, 314, 315, 339, 340, 341, 342, 343, 344, 345, 373, 374, 375, 376, 377, 378, 379, 380, 387, 427, 428, 429, 430, 431, 432, 433, 434, 435, and 436.

One skilled in the art will realize that the monomer-favoring variants of the present invention may be used in many different protein types and sizes. Not only are the variants of the present invention applicable to IgGs and other isotypes of humans and other species, but also the variants have utility in Fc domains, CH3 domains, fragments thereof and polypeptides comprising these polypeptides. For Fcs that dimerize through a CH4 domain, or other domain, the variants of the present invention have utility in those domains that provide dimerization. The variants also have utility in any immunoglobulin domain that forms dimers and in any polypeptide comprising such immunoglobulin domain.

The Fc polypeptides and fusion proteins comprising the monomeric Fc polypeptides in the present invention have many useful properties, which include but are not limited to, an increased monomer content, smaller size, and fewer disulfide forming cysteine residues. Smaller proteins are known to have an increased ability to penetrate tumors (Yokota, et al., 1992, Cancer Res 52:3402-3408; Smith, 2001, Curr Opin Investig Drugs 2:1314-1319, incorporated by reference) and are more readily delivered to the lungs (Bitonti, et al., 2004, Proc Natl Acad Sci USA 101:9763-9768, entirely incorporated by reference). Smaller proteins with fewer disulfide bonds are more likely to be produced efficiently in bacterial expression systems than larger proteins with more disulfide bonds. An additional aspect is that, on a per mole basis, solutions with smaller solutes have a lower viscosity than larger solutes. The viscosity of injected therapeutics must be controlled (Shire, et al., 2004, J Pharm Sci 93:1390-1402, entirely incorporated by reference). Whereas additives can be included to increase the viscosity, the viscosity is less readily decreased. High viscosity leads to problems with injection as well as in manufacturing, especially during filtration. Using smaller proteins in diagnostic imaging is also useful with radiolabels with relatively short half-lives, like technetium and fluorine (Kortt, et al., 2001, Biomol Eng 18:95-108, entirely incorporated by reference). In this case the imaging quality is optimized when the half-life of the radioisotope is matched to the in vivo half-life of its proteinaceous binding partner. Small, single-chain, VL and VH fusions have been created and illustrate the benefits of their small size (Shan et al., 1999, J Immunol 162:6589-6595; Wu et al., 2001, Protein Eng 14:1025-1033; Kortt et al., 2001, Biomol Eng 18:95-108; Peipp, et al., 2004, J Immunol Methods 285:265-280, all entirely incorporated by reference). The Fc mutants of the present invention will have these benefits compared to the dimeric Fc domains.

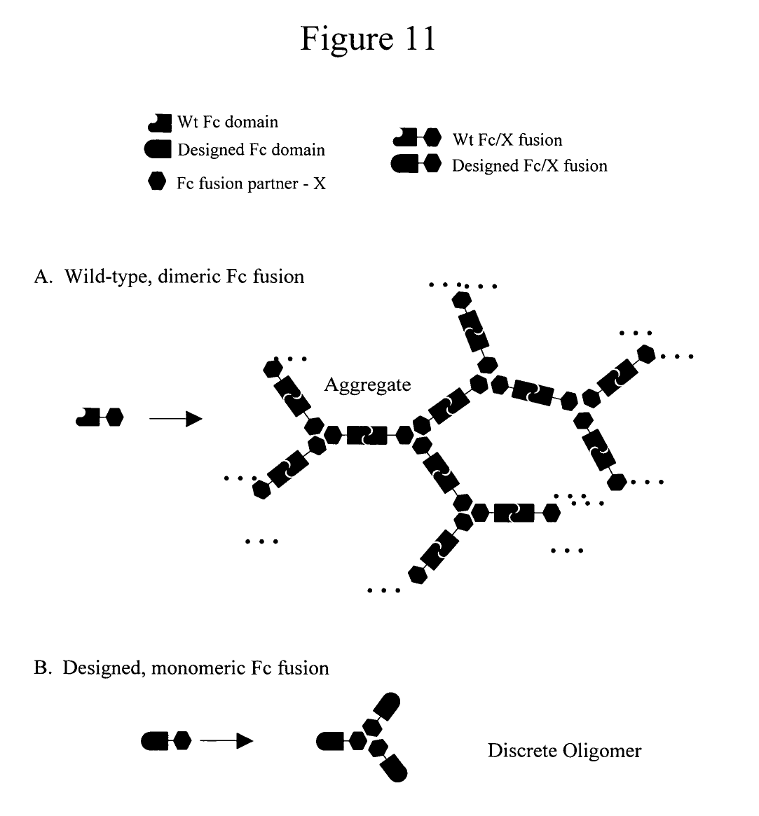

One aspect of the present invention is that it allows the construction of fusions of an Fc domain to proteins (fusion partners) that are not monomeric. The problem of fusing a dimeric Fc to a partner that is a dimer or higher order oligomer is shown in FIG. 11A. If a wild type dimeric Fc domain is fused to a protein that oligomerizes, uncontrolled multimerization occurs leading to protein aggregation. Although this infinite multimerization may be useful to design supramolecular complexes (Yeates and Padilla, 2002 Curr Opin Struc Bio, 12(4): 464-470, entirely incorporated by reference), this uncontrolled multimerization is undesirable for protein therapeutics. If a monomeric Fc domain is fused to a protein that oligomerizes, the oligomerization stops at the natural oligomerization state of the fusion partner (FIG. 11B). Therefore, one aspect of the current invention is to create useful Fc fusions to a protein that is an oligomer, said fusion protein having a reduced tendency to aggregate. Although fusing a protein with an oligomerization states greater than one to monomeric Fc domains is particularly advantageous compared to fusing the partner to a dimeric Fc, any polypeptide may be linked to a monomeric Fc regardless of its oligomeric state.

Another aspect of the present invention is that the monomeric state of the Fc domain will be useful in inhibiting cellular processes that are activated by oligomerization. Some examples occur in the receptor tyrosine kinase family of proteins (Siegal, G J, Agranoff B W, Albers, R W, Fisher S K and Uhler M D. (1999) Basic Neurochemistry, Molecular, Cellular and Medical Aspects. Lippincott, Williams and Wilkens Publisher Philadelphia, entirely incorporated by reference) and in the G-protein coupled receptors (Grant, et al., 2004, J Biol Chem 279:36179-36183, entirely incorporated by reference). For example, platelet-derived growth factor is a dimer, which activates its receptor by binding two receptor molecules and cross-linking them. Receptor ligands that are altered to not form dimers could be linked to a monomeric Fc domain and retain their monomeric nature. The receptor ligands can be made monomeric by mutations or by removal of a dimerization domain from the receptor-binding domain. The exact choice of Fc fusion partner, i.e., the partner that binds a receptor, will depend on the particular receptor/ligand pair in question. In short, use of a monomeric Fc fusion will allow Fc/FcRn binding and maintenance of the monomeric state of a fusion partner, which could allow the binding, but not activation, of different receptors.

Another aspect of the present invention is that the Fc monomers with reduced or no affinity for each other are less likely to exchange disulfide bonds. The Fc monomers will thus have a reduced tendency to form dimers of dimers or other higher order multimers as seen for example in the following references: Schuurman et al., 2001, Mol Immunol 38:1-8; and Wu et al., 2001, Protein Eng 14:1025-1033, both entirely incorporated by reference; and the formation of higher order multimers leads to problems in formulation of Fc fusion therapeutics and is correlated with the onset of hypotension during intravenous administration (Kroez et al., 2003, Biologicals 31:277-286, entirely incorporated by reference). The present invention can help reduce these problems in formulations and help reduce side effects including hypotension.

The Fc monomer variants of the present invention may be combined with other Fc modifications, including but not limited to modifications that alter effector function or interaction with one or more Fc ligands. Such combination may provide additive, synergistic, or novel properties in antibodies or Fc fusions. In one embodiment, the Fc variants of the present invention may be combined with other known Fc variants (Duncan et al., 1988, Nature 332:563-564; Lund et al., 1991, J Immunol 147:2657-2662; Lund et al., 1992, Mol Immunol 29:53-59; Alegre et al., 1994, Transplantation 57:1537-1543; Hutchins et al., 1995, Proc Natl Acad Sci USA 92:11980-11984; Jefferis et al., 1995, Immunol Lett 44:111-117; Lund et al., 1995, Faseb J 9:115-119; Jefferis et al., 1996, Immunol Lett 54:101-104; Lund et al., 1996, J Immunol 157:4963-4969; Armour et al., 1999, Eur J Immunol 29:2613-2624; Idusogie et al., 2000, J Immunol 164:4178-4184; Reddy et al., 2000, J Immunol 164:1925-1933; Xu et al., 2000, Cell Immunol 200:16-26; Idusogie et al., 2001, J Immunol 166:2571-2575; Shields et al., 2001, J Biol Chem 276:6591-6604; Jefferis et al., 2002, Immunol Lett 82:57-65; Presta et al., 2002, Biochem Soc Trans 30:487-490; Hinton et al., 2004, J Biol Chem 279:6213-6216; U.S. Pat. No. 5,624,821; U.S. Pat. No. 5,885,573; U.S. Pat. No. 6,194,551; PCT WO 00/42072; PCT WO 99/58572; US 2004/0002587 A1, all entirely incorporated by reference). In an alternate embodiment, the Fc variants of the present invention are entirely incorporated into an antibody or Fc fusion that comprises one or more engineered glycoforms. By “engineered glycoform” as used herein is meant a carbohydrate composition that is covalently attached to an Fc polypeptide, wherein said carbohydrate composition differs chemically from that of a parent Fc polypeptide. Engineered glycoforms may be useful for a variety of purposes, including but not limited to enhancing or reducing effector function. Engineered glycoforms may be generated by a variety of methods known in the art (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473); U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1, all entirely incorporated by reference); (See for example the Potelligent™ technology ofBiowa, Inc., Princeton, N.J. and the GlycoMAb® glycosylation engineering technology of GLYcart biotechnology AG, Zürich, Switzerland). Many of these techniques are based on controlling the level of fucosylated and/or bisecting oligosaccharides that are covalently attached to the Fc region, for example by expressing an Fc polypeptide in various organisms or cell lines, engineered or otherwise (for example Lec-13 CHO cells or rat hybridoma YB2/0 cells), by regulating enzymes involved in the glycosylation pathway (for example FUT8[a 1,6-fucosyltranserase] and/or b1-4-N-acetylglucosaminyl-transferase III [GnTIII]), or by modifying carbohydrate(s) after the Fc polypeptide has been expressed. Engineered glycoform typically refers to the different carbohydrate or oligosaccharide; thus an Fc polypeptide, for example an antibody or Fc fusion, may comprise an engineered glycoform. Alternatively, engineered glycoform may refer to the Fc polypeptide that comprises the different carbohydrate or oligosaccharide. Thus combinations of the Fc variants of the present invention with other Fc modifications, as well as undiscovered Fc modifications, are contemplated with the goal of generating novel antibodies or Fc fusions with optimized properties.

The Fc monomers of the present invention may find use in an antibody. By “antibody of the present invention” as used herein is meant an antibody that comprises an Fc monomer variant of the present invention. The present invention may, in fact, find use in any protein that comprises Fc, and thus application of the Fc variants of the present invention is not limited to antibodies. The Fc variants of the present invention may find use in an Fc fusion. By “Fc fusion of the present invention” as used herein refers to an Fc fusion that comprises an Fc variant of the present invention. Fc fusions may comprise an Fc variant of the present invention operably linked to a cytokine, soluble receptor domain, adhesion molecule, ligand, enzyme, peptide, or other protein or protein domain, and include but are not limited to Fc fusions described in for example, U.S. Pat. No. 5,843,725; U.S. Pat. No. 6,018,026; U.S. Pat. No. 6,291,212; U.S. Pat. No. 6,291,646; U.S. Pat. No. 6,300,099; U.S. Pat. No. 6,323,323; PCT WO 00/24782; and in Chamow et al., 1996, Trends Biotechnol 14:52-60; Ashkenazi et al., 1997, Curr Opin Immunol 9:195-200, all entirely incorporated by reference).

Virtually any antigen may be targeted by the antibodies and fusions of the present invention, including but not limited to the following list of proteins, subunits, domains, motifs, and epitopes belonging to the following list of proteins: CD2; CD3, CD3E, CD4, CD11, CD11a, CD14, CD16, CD18, CD19, CD20, CD22, CD23, CD25, CD28, CD29, CD30, CD32, CD33 (p67 protein), CD38, CD40, CD40, CD52, CD54, CD56, CD80, CD147, GD3, IL-1, IL-1R, IL-2, IL-2R, IL-4, IL-5, IL-6, IL-6R, IL-8, IL-12, IL-15, IL-18, IL-23, interferon alpha, interferon beta, interferon gamma; TNF-alpha, TNFbeta2, TNFc, TNFalphabeta, TNF-RI, TNF-RII, FasL, CD27L, CD30L, 4-1BBL, TRAIL, RANKL, TWEAK, APRIL, BAFF, LIGHT, VEGI, OX40L, TRAIL Receptor-1, A1 Adenosine Receptor, Lymphotoxin Beta Receptor, TACI, BAFF-R, EPO; LFA-3, ICAM-1, ICAM-3, EpCAM, integrin beta1, integrin beta2, integrin alpha4/beta7, integrin alpha2, integrin alpha3, integrin alpha4, integrin alpha5, integrin alpha6, integrin alphav, alphaVbeta3 integrin, FGFR-3, Keratinocyte Growth Factor, VLA-1, VLA-4, L-selectin, anti-Id, E-selectin, HLA, HLA-DR, CTLA-4, T cell receptor, B7-1, B7-2, VNRintegrin, TGFbeta1, TGFbeta2, eotaxin1, BLyS (B-lymphocyte Stimulator), complement C5, IgE, factor VII, CD64, CBL, NCA 90, EGFR (ErbB-1), Her2/neu (ErbB-2), Her3 (ErbB-3), Her4 (ErbB-4), Tissue Factor, VEGF, VEGFR, endothelin receptor, VLA-4, Hapten NP-cap or NIP-cap, T cell receptor alpha/beta, E-selectin, digoxin, placental alkaline phosphatase (PLAP) and testicular PLAP-like alkaline phosphatase, transferrin receptor, Carcinoembryonic antigen (CEA), CEACAM5, HMFG PEM, mucin MUC1, MUC18, Heparanase I, human cardiac myosin, tumor-associated glycoprotein-72 (TAG-72), tumor-associated antigen CA 125, Prostate specific membrane antigen (PSMA), High molecular weight melanoma-associated antigen (HMW-MAA), carcinoma-associated antigen, Gcoprotein IIb/IIIa (GPIIb/IIIa), tumor-associated antigen expressing Lewis Y related carbohydrate, human cytomegalovirus (HCMV) gH envelope glycoprotein, HIV gp 120, HCMV, respiratory syncital virus RSV F, RSVF Fgp, VNRintegrin, IL-8, cytokeratin tumor-associated antigen, Hep B gp 120, CMV, gpIIbIIIa, HIV IIIB gp 120 V3 loop, respiratory syncytial virus (RSV) Fgp, Herpes simplex virus (HSV) gD glycoprotein, HSV gB glycoprotein, HCMV gB envelope glycoprotein, and Clostridium perfringens toxin.

One skilled in the art will appreciate that the aforementioned list of targets refers not only to specific proteins and biomolecules, but the biochemical pathway or pathways that comprise them. For example, reference to CTLA-4 as a target antigen implies that the ligands and receptors that make up the T cell co-stimulatory pathway, including CTLA-4, B7-1, B7-2, CD28, and any other undiscovered ligands or receptors that bind these proteins, are also targets. Thus target as used herein refers not only to a specific biomolecule, but the set of proteins that interact with said target and the members of the biochemical pathway to which said target belongs. One skilled in the art will further appreciate that any of the aforementioned target antigens, the ligands or receptors that bind them, or other members of their corresponding biochemical pathway, may be operably linked to the Fc variants of the present invention in order to generate an Fc fusion. Thus for example, an Fc fusion that targets EGFR could be constructed by operably linking an Fc variant to EGF, TGFa, or any other ligand, discovered or undiscovered, that binds EGFR. Accordingly, an Fc variant of the present invention could be operably linked to EGFR in order to generate an Fc fusion that binds EGF, TGFa, or any other ligand, discovered or undiscovered, that binds EGFR. Thus virtually any polypeptide, whether a ligand, receptor, or some other protein or protein domain, including but not limited to the aforementioned targets and the proteins that compose their corresponding biochemical pathways, may be operably linked to the Fc variants of the present invention to develop an Fc fusion.

A number of antibodies and Fc fusions that are approved for use, in clinical trials, or in development may benefit from the Fc variants of the present invention. Said antibodies and Fc fusions may be herein referred to as “clinical products and candidates”. Thus in a preferred embodiment, the Fc variants of the present invention may find use in a range of clinical products and candidates. For example, a number of antibodies that target CD20 may benefit from the Fc variants of the present invention. For example the Fc variants of the present invention may find use in an antibody that is substantially similar to rituximab (Rituxan®), Biogenldec/Genentech/Roche) (see for example U.S. Pat. No. 5,736,137, entirely incorporated by reference), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), and HumaLYM (Intracel). A number of antibodies that target members of the family of epidermal growth factor receptors, including EGFR (ErbB-1), Her2/neu (ErbB-2), Her3 (ErbB-3), Her4 (ErbB-4), may benefit from the Fc variants of the present invention. For example the Fc variants of the present invention may find use in an antibody that is substantially similar to trastuzumab (Herceptin®, Genentech) (see for example U.S. Pat. No. 5,677,171, entirely incorporated by reference), a humanized anti-Her2/neu antibody approved to treat breast cancer; pertuzumab (rhuMab-2C4, Omnitarg™), currently being developed by Genentech; an anti-Her2 antibody described in U.S. Pat. No. 4,753,894, entirely incorporated by reference; cetuximab (Erbitux®, Imclone) (U.S. Pat. No. 4,943,533 and PCT WO 96/40210, both entirely incorporated by reference), a chimeric anti-EGFR antibody in clinical trials for a variety of cancers; ABX-EGF (U.S. Pat. No. 6,235,883, entirely incorporated by reference), currently being developed by Abgenix/lmmunex/Amgen; HuMax-EGFr (U.S. Ser. No. 10/172,317, entirely incorporated by reference), currently being developed by Genmab; 425, EMD55900, EMD62000, and EMD72000 (Merck KGaA) (U.S. Pat. No. 5558864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83, all entirely incorporated by reference); ICR62 (Institute of Cancer Research) (PCT WO 95/20045; Modjtahedi et al., 1993, J. Cell Biophys. 1993, 22(1-3):129-46; Modjtahedi et al., 1993, Br J Cancer. 1993, 67(2):247-53; Modjtahedi et al, 1996, Br J Cancer, 73(2):228-35; Modjtahedi et al, 2003, Int J Cancer, 105(2):273-80, all entirely incorporated by reference); TheraCIM hR3 (YM Biosciences, Canada and Centro de Immunologia Molecular, Cuba (U.S. Pat. No. 5,891,996; U.S. Pat. No. 6,506,883; Mateo et al, 1997, Immunotechnology, 3(1):71-81, all entirely incorporated by reference); mAb-806 (Ludwig Institue for Cancer Research, Memorial Sloan-Kettering) (Jungbluth et al. 2003, Proc Natl Acad Sci U S A. 100(2):639-44, entirely incorporated by reference); KSB-102 (KS Biomedix); MR1-1 (IVAX, National Cancer Institute) (PCTWO 0162931A2, entirely incorporated by reference); and SC100 (Scancell) (PCT WO 01/88138, entirely incorporated by reference). In another preferred embodiment, the Fc variants of the present invention may find use in alemtuzumab (Campath®, Millenium), a humanized monoclonal antibody currently approved for treatment of B-cell chronic lymphocytic leukemia. The Fc variants of the present invention may find use in a variety of antibodies or Fc fusions that are substantially similar to other clinical products and candidates, including but not limited to muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering A G, gemtuzumab ozogamicin (Mylotarg®), an anti-CD33 (p67 protein) antibody developed by Celltech/Wyeth, alefacept (Amevive®), an anti-LFA-3 Fc fusion developed by Biogen), abciximab (ReoPro®), developed by Centocor/Lilly, basiliximab (Simulect®), developed by Novartis, palivizumab (Synagis®), developed by Medlmmune, infliximab (Remicade®), an anti-TNFalpha antibody developed by Centocor, adalimumab (Humira®), an anti-TNFalpha antibody developed by Abbott, Humicade™ or Humira®, an anti-TNFalpha antibody developed by Celltech, etanercept (Enbrel®), an anti-TNFalpha Fc fusion developed by Immunex/Amgen, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomab (R1549, 90Y-muHMFG1), an anti-MUC1 In development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegren® (natalizumab), an anti-alpha4-beta-1 (VLA-4) and alpha-4-beta-7 antibody being developed by Biogen, VLA-1 mAb, an anti-VLA1 integrin antibody being developed by Biogen, LTBR mAb, an anti-lymphotoxin beta receptor (LTBR) antibody being developed by Biogen, CAT-152, an anti-TGFb2 antibody being developed by Cambridge Antibody Technology, J695, an anti-IL-12 antibody being developed by Cambridge Antibody Technology and Abbott, CAT-192, an anti-TGFb1 antibody being developed by Cambridge Antibody Technology and Genzyme, CAT-213, an anti-Eotaxin 1 antibody being developed by Cambridge Antibody Technology, LymphoStat-B™ an anti-Blys antibody being developed by Cambridge Antibody Technology and Human Genome Sciences Inc., TRAIL-R1mAb, an anti-TRAIL-R1 antibody being developed by Cambridge Antibody Technology and Human Genome Sciences, Inc., Avastin™ (bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair™ (Omalizumab), an anti-IgE antibody being developed by Genentech, Raptiva™ (Efalizumab), an anti-CD11 a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-I15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide™ (labetuzumab), an anti-carcinoembryonic antigen (CEA) antibody being developed by Immunomedics, LymphoCide™ (Epratuzumab), an anti-CD22 antibody being developed by Immunomedics, AFP-Cide™, being developed by Immunomedics, MyelomaCide™, being developed by Immunomedics, LkoCide, being developed by Immunomedics, ProstaCide, being developed by Immunomedics, MDX-010, an anti-CRLA4 antibody being developed by Medarex, MDX-060, an anti-CD30 antibody being developed by Medarex, MDX-070 being developed by Medarex, MDX-018 being developed by Medarex, Osidem™ (IDM-1), and anti-Her2 antibody being developed by Medarex and Immuno-Designed Molecules, HuMax™-CD4, an anti-CD4 antibody being developed by Medarex and Genmab, HuMax-IL15, an anti-IL15 antibody being developed by Medarex and Genmab, CNTO 148, an anti-TNFa antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion®(visilizumab), an anti-CD3 antibody being developed by Protein Design Labs, HuZAF™, an anti-gamma interferon antibody being developed by Protein Design Labs, Anti-α5β1 Integrin, being developed by Protein Design Labs, anti-IL-12, being developed by Protein Design Labs, ING-1, an anti-Ep-CAM antibody being developed by Xoma, and MLN01, an anti-Beta2 integrin antibody being developed by Xoma.

Application of the Fc monomers of the present invention to the aforementioned antibody and Fc fusion clinical products and candidates is not meant to be constrained to their precise composition. The Fc monomers of the present invention may be entirely incorporated into the aforementioned clinical candidates and products, or into antibodies and Fc fusions that are substantially similar to them. The Fc monomer variants of the present invention may be entirely incorporated into versions of the aforementioned clinical candidates and products that are humanized, affinity matured, engineered, or modified in some other way. Furthermore, the entire polypeptide of the aforementioned clinical products and candidates need not be used to construct a new antibody or Fc fusion that incorporates the Fc monomer variants of the present invention; for example only the variable region of a clinical product or candidate antibody, a substantially similar variable region, or a humanized, affinity matured, engineered, or modified version of the variable region may be used. In another embodiment, the Fc monomer variants of the present invention may find use in an antibody or Fc fusion that binds to the same epitope, antigen, ligand, or receptor as one of the aforementioned clinical products and candidates.

The Fc monomers of the present invention may find use in a wide range of antibody and Fc fusion products. In one embodiment the antibody or Fc fusion of the present invention is a therapeutic, a diagnostic, or a research reagent, preferably a therapeutic. Alternatively, the antibodies and Fc fusions of the present invention may be used for agricultural or industrial uses. In an alternate embodiment, the Fc variants of the present invention compose a library that may be screened experimentally. This library may be a list of nucleic acid or amino acid sequences, or may be a physical composition of nucleic acids or polypeptides that encode the library sequences. The Fc variant may find use in an antibody composition that is monoclonal or polyclonal. The antibodies and Fc fusions of the present invention may be agonists, antagonists, neutralizing, inhibitory, or stimulatory. In a preferred embodiment, the antibodies and Fc fusions of the present invention are used to kill target cells that bear the target antigen, for example cancer cells. In an alternate embodiment, the antibodies and Fc fusions of the present invention are used to block, antagonize, or agonize the target antigen, for example for antagonizing a cytokine or cytokine receptor. In an alternately preferred embodiment, the antibodies and Fc fusions of the present invention are used to block, antagonize, or agonize the target antigen and kill the target cells that bear the target antigen.

The Fc monomer variants of the present invention may be used for various therapeutic purposes. In a preferred embodiment, the Fc variant proteins are administered to a patient to treat an antibody-related disorder. A “patient” for the purposes of the present invention includes both humans and other animals, preferably mammals and most preferably humans. Thus the antibodies and Fc fusions of the present invention have both human therapy and veterinary applications. In the preferred embodiment the patient is a mammal, and in the most preferred embodiment the patient is human. The term “treatment” in the present invention is meant to include therapeutic treatment, as well as prophylactic, or suppressive measures for a disease or disorder. Thus, for example, successful administration of an antibody or Fc fusion prior to onset of the disease results in treatment of the disease. As another example, successful administration of an optimized antibody or Fc fusion after clinical manifestation of the disease to combat the symptoms of the disease comprises treatment of the disease. “Treatment” also encompasses administration of an optimized antibody or Fc fusion protein after the appearance of the disease in order to eradicate the disease. Successful administration of an agent after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented. By “antibody related disorder” or “antibody responsive disorder” or “condition” or “disease” herein are meant a disorder that may be ameliorated by the administration of a pharmaceutical composition comprising an antibody or Fc fusion of the present invention. Antibody related disorders include but are not limited to autoimmune diseases, immunological diseases, infectious diseases, inflammatory diseases, neurological diseases, fibrotic diseases, oncological and neoplastic diseases including cancer. By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer. Furthermore, the Fc variants of the present invention may be used to treat conditions including but not limited to congestive heart failure (CHF), vasculitis, rosecea, acne, eczema, myocarditis and other conditions of the myocardium, systemic lupus erythematosus, diabetes, spondylopathies, synovial fibroblasts, and bone marrow stroma; bone loss; Paget's disease, osteoclastoma; multiple myeloma; breast cancer; disuse osteopenia; malnutrition, periodontal disease, Gaucher's disease, Langerhans' cell histiocytosis, spinal cord injury, acute septic arthritis, osteomalacia, Cushing's syndrome, monoostotic fibrous dysplasia, polyostotic fibrous dysplasia, periodontal reconstruction, and bone fractures; sarcoidosis; multiple myeloma; osteolytic bone cancers, breast cancer, lung cancer, kidney cancer and rectal cancer; bone metastasis, bone pain management, and humoral malignant hypercalcemia, ankylosing spondylitisa and other spondyloarthropathies; transplantation rejection, viral infections, hematologic neoplasisas and neoplastic-like conditions for example, Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia, tumors of the central nervous system, e.g., brain tumors (glioma, neuroblastoma, astrocytoma, medulloblastoma, ependymoma, and retinoblastoma), solid tumors (nasopharyngeal cancer, basal cell carcinoma, pancreatic cancer, cancer of the bile duct, Kaposi's sarcoma, testicular cancer, uterine, vaginal or cervical cancers, ovarian cancer, primary liver cancer or endometrial cancer, and tumors of the vascular system (angiosarcoma and hemagiopericytoma). Other conditions that may be treated using the monomeric Fc variants of the present invention include but are not limited to, arthritis, psoriatic arthritis, ankylosing spondylitis, spondyloarthritis, spondyloarthropathies, rheumatoid arthritis, juvenile rheumatoid arthritis, juvenile idiopathic arthritis, reactive arthritis (Reiter Syndrome) scleroderma, Sjogren's syndrome, keratoconjunctivitis, keratoconjunctivitis sicca, TNF-receptor associated periodic syndrome (TRAPS), periodic fever, periprosthetic osteolysis, apthous stomatitis, pyoderma gangrenosum, uveitis, reticulohistiocytosis, inflammatory bowel diseases, sepsis and septic shock, Crohn's Disease, psoriasis, autoimmune thyroiditis, dermatitis, atopic dermatitis, eczematous dermatitis)graft versus host disease (GVHD), hematologic malignancies, such as multiple myeloma (MM), refractory MM, Waldenstrom's macroglobulinemia, myelodysplastic syndrome (MDS) acute myelogenous leukemia (AML); solid tumor malignancies, such as ovarian carcinoma, melanoma, renal cell carcinoma; and the inflammation associated with tumors, pain, including spinal disk pain, chronic lower back pain chronic neck pain, pain due to bone metastasis, pain and swelling after molar extraction, neurological conditions and neural damage conditions such as peripheral nerve injury, demyelinating diseases, adrenoleukodystrophy, X-linked adrenoleukodystrophy (X-ALD), the childhood cerebral form (CCER) and the adult form, adrenomyeloneuropathy (AMN), adrenoleukodystrophy, sciatica, autoimmune sensorineural hearing loss, chronic inflammatory demyelinating polyneuropathy (CIDP), Alzheimers disease, Parkinson's disease, diabetes, insulin resistance, insulin sensitivity, Syndrome X, Wegener's Granulomatosis, dermatomyositis, histicytosis, polymyositis, cancer cachexia, temporomandibular disorders, refractory ocular sarcoidosis, sarcoidosis, behcet's, churg-strauss syndrome, asthma, idiopatic pneumonia following bone marrow transplantation, systemic lupus erythematosus (SLE), lupus nephritis, multiple sclerosis (MS), amyotrophic lateral sclerosis (ALS) myasthenia gravis, atherosclerosis, polyneuropathy, orangomegaly, endocrinopathy, M protein, skin changes (POEMS syndrome), Sneddon-Wilkinson disease, necrotizing crescentic glomerulonephritis, renal amyloidosis, AA amyloidosis, erythema nodosum leprosum (ENL), chronic kidney disease, malnutrition, inflammation and atherosclerosis (MIA) syndrome, chronic obstructive pulmonary disease (COPD), pulmonary fibrosis, endometriosis, idiopathic thrombocytopenic purpura (ITP), AIDS, HIV disease and related conditions, including tuberculosis (TB) in AIDS patients, inflammation and cancer (e.g. Kaposi's Sarcoma, HIV retinopathy, uveitis, P jiroveci pneumonia (PCP), Pneumocystis choroiditis, HIV-associated lymphoma), alopecia areata, allergic responses due to arthropod bite reactions, aphthous ulcer, iritis, conjunctivitis, keratoconjunctivitis, ulcerative colitis, asthma, allergic asthma, cutaneous lupus erythematosus, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens Johnson syndrome, idiopathic sprue, lichen planus, Graves ophthalmopathy, sarcoidosis, primary biliary cirrhosis, and interstitial lung fibrosis.

In one embodiment, an antibody or Fc fusion of the present invention is administered to a patient having a disease involving inappropriate expression of a protein. Within the scope of the present invention this is meant to include diseases and disorders characterized by aberrant proteins, due for example to alterations in the amount of a protein present, the presence of a mutant protein, or both. An overabundance may be due to any cause, including but not limited to overexpression at the molecular level, prolonged or accumulated appearance at the site of action, or increased activity of a protein relative to normal. Included within this definition are diseases and disorders characterized by a reduction of a protein. This reduction may be due to any cause, including but not limited to reduced expression at the molecular level, shortened or reduced appearance at the site of action, mutant forms of a protein, or decreased activity of a protein relative to normal. Such an overabundance or reduction of a protein can be measured relative to normal expression, appearance, or activity of a protein, and said measurement may play an important role in the development and/or clinical testing of the antibodies and Fc fusions of the present invention.

In one embodiment, an antibody or Fc fusion of the present invention is the only therapeutically active agent administered to a patient. Alternatively, the antibody or Fc fusion of the present invention is administered in combination with one or more other therapeutic agents, including but not limited to cytotoxic agents, chemotherapeutic agents, cytokines, growth inhibitory agents, anti-hormonal agents, kinase inhibitors, anti-angiogenic agents, cardioprotectants, or other therapeutic agents. Such molecules are suitably present in combination in amounts that are effective for the purpose intended. The skilled medical practitioner can determine empirically the appropriate dose or doses of other therapeutic agents useful herein. The antibodies and Fc fusions of the present invention may be administered concomitantly with one or more other therapeutic regimens. For example, an antibody or Fc fusion of the present invention may be administered to the patient along with chemotherapy, radiation therapy, or both chemotherapy and radiation therapy. In one embodiment, the antibody or Fc fusion of the present invention may be administered in conjunction with one or more antibodies or Fc fusions, which may or may not comprise an Fc variant of the present invention.

In one embodiment, the antibodies and Fc fusions of the present invention are administered with a chemotherapeutic agent. By “chemotherapeutic agent” as used herein is meant a chemical compound useful in the treatment of cancer. Examples of chemotherapeutic agents include but are not limited to alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (Taxol®, Bristol-Myers Squibb Oncology, Princeton, N.J.) and docetaxel (Taxotere®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; thymidylate synthase inhibitor (such as Tomudex); cox-2 inhibitors, such as celicoxib (Celebrex®) or MK-0966 (Vioxx®); and pharmaceutically acceptable salts, acids or derivatives of any of the above. Also included in this definition are anti-hormonal agents that act to regulate or inhibit hormone action on tumors such as anti estrogens including for example tamoxifen, raloxifene, aromatase inhibiting 4(5)-imidazoles, 4-hydroxytamoxifen, trioxifene, keoxifene, LY 117018, onapristone, and toremifene (Fareston®); and anti-androgens such as flutamide, nilutamide, bicalutamide, leuprolide, and goserelin; and pharmaceutically acceptable salts, acids or derivatives of any of the above.

A chemotherapeutic or other cytotoxic agent may be administered as a prodrug. By “prodrug” as used herein is meant a precursor or derivative form of a pharmaceutically active substance that is less cytotoxic to tumor cells compared to the parent drug and is capable of being enzymatically activated or converted into the more active parent form. See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985, both entirely incorporated by reference. The prodrugs that may find use with the present invention include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the present invention include but are not limited to any of the aforementioned chemotherapeutic agents.

The antibodies and Fc fusions of the present invention may be combined with other therapeutic regimens. For example, in one embodiment, the patient to be treated with the antibody or Fc fusion may also receive radiation therapy. Radiation therapy can be administered according to protocols commonly employed in the art and known to the skilled artisan. Such therapy includes but is not limited to cesium, iridium, iodine, or cobalt radiation. The radiation therapy may be whole body irradiation, or may be directed locally to a specific site or tissue in or on the body, such as the lung, bladder, or prostate. Typically, radiation therapy is administered in pulses over a period of time from about 1 to 2 weeks. The radiation therapy may, however, be administered over longer periods of time. For instance, radiation therapy may be administered to patients having head and neck cancer for about 6 to about 7 weeks. Optionally, the radiation therapy may be administered as a single dose or as multiple, sequential doses. The skilled medical practitioner can determine empirically the appropriate dose or doses of radiation therapy useful herein. In accordance with another embodiment of the invention, the antibody or Fc fusion of the present invention and one or more other anti-cancer therapies are employed to treat cancer cells ex vivo. It is contemplated that such ex vivo treatment may be useful in bone marrow transplantation and particularly, autologous bone marrow transplantation. For instance, treatment of cells or tissue(s) containing cancer cells with antibody or Fc fusion and one or more other anti-cancer therapies, such as described above, can be employed to deplete or substantially deplete the cancer cells prior to transplantation in a recipient patient. It is of course contemplated that the antibodies and Fc fusions of the invention can be employed in combination with still other therapeutic techniques such as surgery.

In an alternate embodiment, the antibodies and Fc fusions of the present invention are administered with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -Il; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

A variety of other therapeutic agents may find use for administration with the antibodies and Fc fusions of the present invention. In one embodiment, the antibody or Fc fusion is administered with an anti-angiogenic agent. By “anti-angiogenic agent” as used herein is meant a compound that blocks, or interferes to some degree, the development of blood vessels. The anti-angiogenic factor may, for instance, be a small molecule or a protein, for example an antibody, Fc fusion, or cytokine, that binds to a growth factor or growth factor receptor involved in promoting angiogenesis. The preferred anti-angiogenic angiogenic factor herein is an antibody that binds to Vascular Endothelial Growth Factor (VEGF). In an alternate embodiment, the antibody or Fc fusion is administered with a therapeutic agent that induces or enhances adaptive immune response, for example an antibody that targets CTLA-4. In an alternate embodiment, the antibody or Fc fusion is administered with a tyrosine kinase inhibitor. By “tyrosine kinase inhibitor” as used herein is meant a molecule that inhibits to some extent tyrosine kinase activity of a tyrosine kinase. Examples of such inhibitors include but are not limited to quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo(2,3-d) pyrimidines; curcumin (diferuloyl methane, 4,5-bis (4-fluoroanilino)phthalimide); tyrphostines containing nitrothiophene moieties; PD-0183805 (Wamer-Lambert); antisense molecules (e.g. those that bind to ErbB-encoding nucleic acid); quinoxalines (U.S. Pat. No. 5,804,396); tryphostins (U.S. Pat. No. 5,804,396, entirely incorporated by reference); ZD6474 (Astra Zeneca); PTK-787 (Novartis/Schering A G); pan-ErbB inhibitors such as C1-1033 (Pfizer); Affinitac (ISIS 3521; Isis/Lilly); Imatinib mesylate (STI571,Gleevec®; Novartis); PKI 166 (Novartis); GW2016 (GlaxoSmithKline); C1-1033 (Pfizer); EKB-569 (Wyeth); Semaxinib (Sugen); ZD6474 (AstraZeneca); PTK-787 (Novartis/Schering AG); INC-1C11 (Imclone); and other agents such as gefitinib (Iressa®, ZD1839, AstraZeneca), and OSI-774 (Tarceva®, OSI Pharmaceuticals/Genentech); or as described in any of the following patent publications: U.S. Pat. No. 5,804,396; PCT WO 99/09016; PCT WO 98/43960; PCT WO 97/38983; PCT WO 99/06378; PCT WO 99/06396; PCT WO 96/30347; PCT WO 96/33978; PCT W096/3397; PCT WO 96/33980, all hereby entirely incorporated by reference.

A variety of linkers may find use in the present invention to generate Fc fusions (see definition above) or antibody- or Fc fusion- conjugates (see definition below). By “linker”, “linker sequence”, “spacer”, “tethering sequence” or grammatical equivalents thereof, herein is meant a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. Choosing a suitable linker for a specific case where two polypeptide chains are to be connected depends on various parameters, including but not limited to the nature of the two polypeptide chains (e.g., whether they naturally oligomerize), the distance between the N— and the C-termini to be connected if known, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 30 amino acid residues. Preferably, the linker is from about 1 to 30 amino acids in length, with linkers of 1, 2, 3, 4, 5, 6, 7, 8, 9, 10, 11, 12, 13, 14, 15, 16, 17, 18 19 and 20 amino acids in length being preferred. In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. Useful linkers include glycine-serine polymers (including, for example, (GS)n, (GSGGS)n (GGGGS)n and (GGGS)n, where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies. Suitable linkers may also be identified by screening databases of known three-dimensional structures for naturally occurring motifs that can bridge the gap between two polypeptide chains. In a preferred embodiment, the linker is not immunogenic when administered in a human patient. Thus linkers may be chosen such that they have low immunogenicity or are thought to have low immunogenicity. For example, a linker may be chosen that exists naturally in a human. In a preferred embodiment the linker has the sequence of the hinge region of an antibody, that is the sequence that links the antibody Fab and Fc regions; alternatively the linker has a sequence that comprises part of the hinge region, or a sequence that is substantially similar to the hinge region of an antibody. Another way of obtaining a suitable linker is by optimizing a simple linker, e.g., (Gly4Ser)n, through random mutagenesis. Alternatively, once a suitable polypeptide linker is defined, additional linker polypeptides can be created to select amino acids that more optimally interact with the domains being linked. Alternatively, a linker may be designed by computational method to interact with another part of the polypeptide. The interactions are designed to help sequester the linker from proteolysis or other degradative processes. Other types of linkers that may be used in the present invention include artificial polypeptide linkers and inteins. In another embodiment, disulfide bonds are designed to link the two molecules. In another embodiment, linkers are chemical cross-linking agents. For example, a variety of bifunctional protein coupling agents may be used, including but not limited to N-succinimidyl-3-(2-pyridyidithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., 1971, Science 238:1098, entirely incorporated by reference. Chemical linkers may enable chelation of an isotope. For example, Carbon-14-labeled 1-isothio-cyanatobenzyl-3-methyldiethylene triaminepentaacetic acid (MX-DTPA) is an exemplary chelating agent for conjugation of radionucleotide to the antibody (see PCT WO 94/11026, entirely incorporated by reference). The linker may be cleavable, facilitating release of the cytotoxic drug in the cell. For example, an acid-labile linker, peptidase-sensitive linker, dimethyl linker or disulfide-containing linker (Chari et al., 1992, Cancer Research 52: 127-131, entirely incorporated by reference) may be used. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use to link the Fc variants of the present invention to a fusion partner to generate an Fc fusion, or to link the antibodies and Fc fusions of the present invention to a conjugate.

In one embodiment, the antibody or Fc fusion of the present invention is conjugated or operably linked to another therapeutic compound, referred to herein as a conjugate. The conjugate may be a cytotoxic agent, a chemotherapeutic agent, a cytokine, an anti-angiogenic agent, a tyrosine kinase inhibitor, a toxin, a radioisotope, or other therapeutically active agent. Chemotherapeutic agents, cytokines, anti-angiogenic agents, tyrosine kinase inhibitors, and other therapeutic agents have been described above, and all of these aforemention therapeutic agents may find use as antibody or Fc fusion conjugates. In an alternate embodiment, the antibody or Fc fusion is conjugated or operably linked to a toxin, including but not limited to small molecule toxins and enzymatically active toxins of bacterial, fungal, plant or animal origin, including fragments and/or variants thereof. Small molecule toxins include but are not limited to calicheamicin, maytansine (U.S. Pat. No. 5,208,020, entirely incorporated by reference), trichothene, and CC1065. In one embodiment of the invention, the antibody or Fc fusion is conjugated to one or more maytansine molecules (e.g. about 1 to about 10 maytansine molecules per antibody molecule). Maytansine may, for example, be converted to May-SS-Me, which may be reduced to May-SH3 and reacted with modified antibody or Fc fusion (Chari et al., 1992, Cancer Research 52: 127-131, entirely incorporated by reference) to generate a maytansinoid-antibody or maytansinoid-Fc fusion conjugate. Another conjugate of interest comprises an antibody or Fc fusion conjugated to one or more calicheamicin molecules. The calicheamicin family of antibiotics are capable of producing double-stranded DNA breaks at sub-picomolar concentrations. Structural analogs of calicheamicin that may be used include but are not limited to g11, a21, a3, N-acetyl-g11, PSAG, and Θ11, (Hinman et al., 1993, Cancer Research 53:3336-3342; Lode et al., 1998, Cancer Research 58:2925-2928; U.S. Pat. No. 5,714,586; U.S. Pat. No. 5,712,374; U.S. Pat. No. 5,264,586; U.S. Pat. No. 5,773,001, all entirely incorporated by reference). Dolastatin 10 analogs such as auristatin E (AE) and monomethylauristatin E (MMAE) may find use as conjugates for the Fc variants of the present invention (Doronina et al., 2003, Nat Biotechnol 21(7):778-84; and Francisco et al., 2003 Blood 102(4):1458-65 , both entirely incorporated by reference). Useful enyzmatically active toxins include but are not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, PCT WO 93/21232, entirely incorporated by reference. The present invention further contemplates a conjugate or fusion formed between an antibody or Fc fusion of the present invention and a compound with nucleolytic activity, for example a ribonuclease or DNA endonuclease such as a deoxyribonuclease (DNase).

In an alternate embodiment, an antibody or Fc fusion of the present invention may be conjugated or operably linked to a radioisotope to form a radioconjugate. A variety of radioactive isotopes are available for the production of radioconjugate antibodies and Fc fusions. Examples include, but are not limited to, At211, I131, I125, Y90, Re186, Re188, Sm153, Bi212, P32, and radioactive isotopes of Lu.

In yet another embodiment, an antibody or Fc fusion of the present invention may be conjugated to a “receptor” (such streptavidin) for utilization in tumor pretargeting wherein the antibody-receptor or Fc fusion-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). In an alternate embodiment, the antibody or Fc fusion is conjugated or operably linked to an enzyme in order to employ Antibody Dependent Enzyme Mediated Prodrug Therapy (ADEPT™). ADEPT may be used by conjugating or operably linking the antibody or Fc fusion to a prodrug-activating enzyme that converts a prodrug (e.g. a peptidyl chemotherapeutic agent, see PCT WO 81/01145, entirely incorporated by reference) to an active anti-cancer drug. See, for example, PCT WO 88/07378 and U.S. Pat. No. 4,975,278, entirely incorporated by reference. The enzyme component of the immunoconjugate useful for ADEPT includes any enzyme capable of acting on a prodrug in such a way so as to covert it into its more active, cytotoxic form. Enzymes that are useful in the method of this invention include but are not limited to alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptides, useful for converting prodrugs that contain D-amino acid substituents; carbohydrate-cleaving enzymes such as .beta.-galactosidase and neuramimidase useful for converting glycosylated prodrugs into free drugs; beta-lactamase useful for converting drugs derivatized with .alpha.-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, for example, Massey, 1987, Nature 328: 457-458, entirely incorporated by reference). Antibody-abzyme and Fc fusion-abzyme conjugates can be prepared for delivery of the abzyme to a tumor cell population.

Other modifications of the antibodies and Fc fusions of the present invention are contemplated herein. For example, the antibody or Fc fusion may be linked to one of a variety of nonproteinaceous polymers, e.g., polyethylene glycol (PEG), polypropylene glycol, polyoxyalkylenes, or copolymers of polyethylene glycol and polypropylene glycol.

Pharmaceutical compositions are contemplated wherein an antibody or Fc fusion of the present invention and one or more therapeutically active agents are formulated. Formulations of the antibodies and Fc fusions of the present invention are prepared for storage by mixing said antibody or Fc fusion having the desired degree of purity with optional pharmaceutically acceptable carriers, excipients or stabilizers (Remington's Pharmaceutical Sciences 16th edition, Osol, A. Ed., 1980, entirely incorporated by reference), in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN®, PLURONIC® EO/PO block copolymers (BASF), or polyethylene glycol (PEG). In a preferred embodiment, the pharmaceutical composition that comprises the antibody or Fc fusion of the present invention is in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic add, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferrably sterile. This is readily accomplished by filtration through sterile filtration membranes or other methods.

The antibodies and Fc fusions disclosed herein may also be formulated as immunoliposomes. A liposome is a small vesicle comprising various types of lipids, phospholipids and/or surfactant that is useful for delivery of a therapeutic agent to a mammal. Liposomes containing the antibody or Fc fusion are prepared by methods known in the art, such as described in Epstein et al., 1985, Proc Natl Acad Sci USA, 82:3688; Hwang et al., 1980, Proc Natl Acad Sci USA, 77:4030; U.S. Pat. No. 4,485,045; U.S. Pat. No. 4,544,545; and PCT WO 97/38731, all entirely incorporated by reference. Liposomes with enhanced circulation time are disclosed in U.S. Pat. No. 5,013,556, entirely incorporated by reference. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484, entirely incorporated by reference).

The antibodies, Fc fusions, and other therapeutically active agents may also be entrapped in microcapsules prepared by methods including but not limited to coacervation techniques, interfacial polymerization (for example using hydroxymethylcellulose or gelatin-microcapsules, or poly(methylmethacylate) microcapsules), colloidal drug delivery systems (for example, liposomes, albumin microspheres, microemulsions, nanoparticles and nanocapsules), and macroemulsions. Such techniques are disclosed in Remington's Pharmaceutical Sciences 16th edition, Osol, A. Ed., 1980, entirely incorporated by reference. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919, entirely incorporated by reference), copolymers of L-glutamic acid and gamma ethyl-L-glutamate, non-degradable ethylene-vinyl acetate, degradable lactic acid-glycolic acid copolymers such as the Lupron Depot® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule entirely incorporated into a matrix of poly-DL-lactide-co-glycolide (PLG).

The concentration of the therapeutically active antibody or Fc fusion of the present invention in the formulation may vary from about 0.1 to 100 weight %. In a preferred embodiment, the concentration of the antibody or Fc fusion is in the range of 0.003 to 1.0 molar. In order to treat a patient, a therapeutically effective dose of the antibody or Fc fusion of the present invention may be administered. By “therapeutically effective dose” herein is meant a dose that produces the effects for which it is administered. The exact dose will depend on the purpose of the treatment, and will be ascertainable by one skilled in the art using known techniques. Dosages may range from 0.01 to 100 mg/kg of body weight or greater, for example 0.1, 1, 10, or 50 mg/kg of body weight, with 1 to 10 mg/kg being preferred. As is known in the art, adjustments for antibody or Fc fusion degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

Administration of the pharmaceutical composition comprising an antibody or Fc fusion of the present invention, preferably in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary (e.g., AERx®) inhalable technology commercially available from Aradigm, or lnhance™ pulmonary delivery system commercially available from Inhale Therapeutics), vaginally, parenterally, rectally, or intraocularly. In some instances, for example for the treatment of wounds, inflammation, etc., the antibody or Fc fusion may be directly applied as a solution or spray. Alternatively, the compositions of the present invention may be infused, perfused or administered via a pump means including but not limitd to an Alzet® pump. As is known in the art, the pharmaceutical composition may be formulated accordingly depending upon the manner of introduction.

The present invention provides methods for producing and screening libraries of Fc variants. The described methods are not meant to constrain the present invention to any particular application or theory of operation. Rather, the provided methods are meant to illustrate generally that one or more Fc variants or one or more libraries of Fc variants may be produced and screened experimentally to obtain optimized Fc variants. Fc variants may be produced and screened in any context, whether as an Fc region as precisely defined herein, a domain or fragment thereof, or a larger polypeptide that comprises Fc such as an antibody or Fc fusion. General methods for antibody molecular biology, expression, purification, and screening are described in Antibody Engineering, edited by Duebel & Kontermann, Springer-Verlag, Heidelberg, 2001; and Hayhurst & Georgiou, 2001, Curr Opin Chem Biol 5:683-689; Maynard & Georgiou, 2000, Annu Rev Biomed Eng 2:339-76; Antibodies: A Laboratory Manual by Harlow & Lane, New York: Cold Spring Harbor Laboratory Press, 1988, all entirely incorporated by reference.

In one embodiment of the present invention, the sequences are used to create nudeic acids that encode the member sequences, and that may then be doned into host cells, expressed and assayed, if desired. Thus, nucleic acids, and particularly DNA, may be made that encode each member protein sequence. These practices are carried out using well-known procedures. For example, a variety of methods that may find use in the present invention are described in Molecular Cloning—A Laboratory Manual, 3rd Ed. (Maniatis, Cold Spring Harbor Laboratory Press, New York, 2001), and Current Protocols in Molecular Biology (John Wiley & Sons), both entirely incorporated by reference. As will be appreciated by those skilled in the art, the generation of exact sequences for a library comprising a large number of sequences is potentially expensive and time consuming. Accordingly, there are a variety of techniques that may be used to efficiently generate libraries of the present invention. Such methods that may find use in the present invention are described or referenced in U.S. Pat. No. 6,403,312; U.S. Ser. Nos. 09/782,004; 09/927,790; 10/101,499; 10/218,102; 10/666,307 and 10/666,311; PCT WO 01/40091; and PCT WO 02/25588, all entirely incorporated by reference. Such methods include but are not limited to gene assembly methods, PCR-based method and methods which use variations of PCR, ligase chain reaction-based methods, pooled oligomer (oligo) methods such as those used in synthetic shuffling, error-prone amplification methods and methods which use oligos with random mutations, classical site-directed mutagenesis methods, cassette mutagenesis, and other amplification and gene synthesis methods. As is known in the art, there are a variety of commercially available kits and methods for gene assembly, mutagenesis, vector subcloning, and the like, and such commercial products find use in the present invention for generating nucleic acids that encode Fc variant members of a library.

The Fc variants of the present invention may be produced by culturing a host cell transformed with nucleic acid, preferably an expression vector, containing nucleic acid encoding the Fc variants, under the appropriate conditions to induce or cause expression of the protein. The conditions appropriate for expression will vary with the choice of the expression vector and the host cell, and will be easily ascertained by one skilled in the art through routine experimentation. A wide variety of appropriate host cells may be used, including but not limited to mammalian cells, bacteria, insect cells, and yeast. For example, a variety of cell lines that may find use in the present invention are described in the ATCC® cell line catalog, available from the American Type Culture Collection, Manassas, Va., entirely incorporated by reference.

In a preferred embodiment, the Fc variants are expressed in mammalian expression systems, including systems in which the expression constructs are introduced into the mammalian cells using virus such as retrovirus or adenovirus. Any mammalian cells may be used, with human, mouse, rat, hamster, and primate cells being particularly preferred. Suitable cells also include known research cells, including but not limited to Jurkat T cells, NIH3T3, CHO, COS, and 293 cells. In an alternately preferred embodiment, library proteins are expressed in bacterial cells. Bacterial expression systems are well known in the art, and include Escherichia coli (E. coli), Bacillus subtilis, Streptococcus cremoris, and Streptococcus lividans. In alternate embodiments, Fc variants are produced in insect cells or yeast cells. In an alternate embodiment, Fc variants are expressed in vitro using cell free translation systems. In vitro translation systems derived from both prokaryotic (e.g. E. coli) and eukaryotic (e.g. wheat germ, rabbit reticulocytes) cells are available and may be chosen based on the expression levels and functional properties of the protein of interest. For example, as appreciated by those skilled in the art, in vitro translation is required for some display technologies, for example ribosome display. In addition, the Fc variants may be produced by chemical synthesis methods.

The nucleic acids that encode the Fc variants of the present invention may be entirely incorporated into an expression vector in order to express the protein. A variety of expression vectors may be utilized for protein expression. Expression vectors may comprise self-replicating extra-chromosomal vectors or vectors which integrate into a host genome. Expression vectors are constructed to be compatible with the host cell type. Thus expression vectors that find use in the present invention include but are not limited to those which enable protein expression in mammalian cells, bacteria, insect cells, yeast, and in in vitro systems. As is known in the art, a variety of expression vectors are available, commercially or otherwise, that may find use in the present invention for expressing Fc variant proteins.

Expression vectors typically comprise a protein operably linked with control or regulatory sequences, selectable markers, any fusion partners, and/or additional elements. By “operably linked” herein is meant that the nucleic acid is placed into a functional relationship with another nucleic acid sequence. Generally, these expression vectors include transcriptional and translational regulatory nucleic acid operably linked to the nucleic acid encoding the Fc variant, and are typically appropriate to the host cell used to express the protein. In general, the transcriptional and translational regulatory sequences may include promoter sequences, ribosomal binding sites, transcriptional start and stop sequences, translational start and stop sequences, and enhancer or activator sequences. As is also known in the art, expression vectors typically contain a selection gene or marker to allow the selection of transformed host cells containing the expression vector. Selection genes are well known in the art and will vary with the host cell used.

Fc variants may be operably linked to a fusion partner to enable targeting of the expressed protein, purification, screening, display, and the like. Fusion partners may be linked to the Fc variant sequence via a linker sequences. The linker sequence will generally comprise a small number of amino acids, typically less than ten, although longer linkers may also be used. Typically, linker sequences are selected to be flexible and resistant to degradation. As will be appreciated by those skilled in the art, any of a wide variety of sequences may be used as linkers. For example, a common linker sequence comprises the amino acid sequence GGGGS. A fusion partner may be a targeting or signal sequence that directs Fc variant protein and any associated fusion partners to a desired cellular location or to the extracellular media. As is known in the art, certain signaling sequences may target a protein to be either secreted into the growth media, or into the periplasmic space, located between the inner and outer membrane of the cell. A fusion partner may also be a sequence that encodes a peptide or protein that enables purification and/or screening. Such fusion partners include but are not limited to polyhistidine tags (His-tags) (for example H6 and H10 or other tags for use with Immobilized Metal Affinity Chromatography (IMAC) systems (e.g. Ni+2 affinity columns)), GST fusions, MBP fusions, Strep-tag, the BSP biotinylation target sequence of the bacterial enzyme BirA, and epitope tags which are targeted by antibodies (for example c-myc tags, flag-tags, and the like). As will be appreciated by those skilled in the art, such tags may be useful for purification, for screening, or both. For example, an Fc variant may be purified using a His-tag by immobilizing it to a Ni+2 affinity column, and then after purification the same His-tag may be used to immobilize the antibody to a Ni+2 coated plate to perform an ELISA or other binding assay (as described below). A fusion partner may enable the use of a selection method to screen Fc variants (see below). Fusion partners that enable a variety of selection methods are well-known in the art, and all of these find use in the present invention. For example, by fusing the members of an Fc variant library to the gene IlIl protein, phage display can be employed (Kay et al., Phage display of peptides and proteins: a laboratory manual, Academic Press, San Diego, Calif., 1996; Lowman et al., 1991, Biochemistry 30:10832-10838; Smith, 1985, Science 228:1315-1317, all entirely incorporated by reference). Fusion partners may enable Fc variants to be labeled. Alternatively, a fusion partner may bind to a specific sequence on the expression vector, enabling the fusion partner and associated Fc variant to be linked covalently or noncovalently with the nucleic acid that encodes them. For example, U.S. Ser. No. 09/642,574; U.S. Ser. No. 10/080,376; U.S. Ser. No. 09/792,630; U.S. Ser. No. 10/023,208; U.S. Ser. No. 09/792,626; U.S. Ser. No. 10/082,671; U.S. Ser. No. 09/953,351; U.S. Ser. No. 10/097,100; U.S. Ser. No. 60/366,658; PCT WO 00/22906; PCT WO 01/49058; PCT WO 02/04852; PCT WO 02/04853; PCT WO 02/08023; PCT WO 01/28702; and PCT WO 02/07466, all entirely incorporated by reference, describe such a fusion partner and techniques that may find use in the present invention.

The methods of introducing exogenous nucleic acid into host cells are well known in the art, and will vary with the host cell used. Techniques include but are not limited to dextran-mediated transfection, calcium phosphate precipitation, calcium chloride treatment, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

In a preferred embodiment, Fc variant proteins are purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of Fc variants. For example, the bacterial proteins A and G bind to the Fc region. Likewise, the bacterial protein L binds to the Fab region of some antibodies, as of course does the antibody's target antigen. Purification can often be enabled by a particular fusion partner. For example, Fc variant proteins may be purified using glutathione resin if a GST fusion is employed, Ni+2 affinity chromatography if a His-tag is employed, or immobilized anti-flag antibody if a flag-tag is used. For general guidance in suitable purification techniques, see Protein Purification: Principles and Practice, 3rd Ed., Scopes, Springer-Verlag, N.Y., 1994, entirely incorporated by reference. The degree of purification necessary will vary depending on the screen or use of the Fc variants. In some instances no purification is necessary. For example in one embodiment, if the Fc variants are secreted, screening may take place directly from the media. As is well known in the art, some methods of selection do not involve purification of proteins. Thus, for example, if a library of Fc variants is made into a phage display library, protein purification may not be performed.

Fc variants may be screened using a variety of methods, including but not limited to those that use in vitro assays, in vivo and cell-based assays, and selection technologies. Automation and high-throughput screening technologies may be utilized in the screening procedures. Screening may employ the use of a fusion partner or label. The use of fusion partners has been discussed above. By “labeled” herein is meant that the Fc variants of the invention have one or more elements, isotopes, or chemical compounds attached to enable the detection in a screen. In general, labels fall into three classes: a) immune labels, which may be an epitope entirely incorporated as a fusion partner that is recognized by an antibody, b) isotopic labels, which may be radioactive or heavy isotopes, and c) small molecule labels, which may include fluorescent and calorimetric dyes, or molecules such as biotin that enable other labeling methods. Labels may be entirely incorporated into the compound at any position and may be entirely incorporated in vitro or in vivo during protein expression.

In a preferred embodiment, the functional and/or biophysical properties of Fc variants are screened in an in vitro assay. In vitro assays may allow a broad dynamic range for screening properties of interest. Properties of Fc variants that may be screened include but are not limited to stability, solubility, and affinity for Fc ligands, for example FcgRs. Multiple properties may be screened simultaneously or individually. Proteins may be purified or unpurified, depending on the requirements of the assay. In one embodiment, the screen is a qualitative or quantitative binding assay for binding of Fc variants to a protein or nonprotein molecule that is known or thought to bind the Fc variant. In a preferred embodiment, the screen is a binding assay for measuring binding to the antibody's or Fc fusions' target antigen. In an alternately preferred embodiment, the screen is an assay for binding of Fc variants to an Fc ligand, including but are not limited to the family of FcgRs, the neonatal receptor FcRn, the complement protein C1q, and the bacterial proteins A and G. Said Fc ligands may be from any organism, with humans, mice, rats, rabbits, and monkeys preferred. Binding assays can be carried out using a variety of methods known in the art, including but not limited to FRET (Fluorescence Resonance Energy Transfer) and BRET (Bioluminescence Resonance Energy Transfer)-based assays, AlphaScreen™ (Amplified Luminescent Proximity Homogeneous Assay), Scintillation Proximity Assay, ELISA (Enzyme-Linked Immunosorbent Assay), SPR (Surface Plasmon Resonance, also known as BIACORE®), isothermal titration calorimetry, differential scanning calorimetry, gel electrophoresis, and chromatography including gel filtration. These and other methods may take advantage of some fusion partner or label of the Fc variant. Assays may employ a variety of detection methods including but not limited to chromogenic, fluorescent, luminescent, or isotopic labels.

The biophysical properties of Fc variant proteins, for example stability and solubility, may be screened using a variety of methods known in the art. Protein stability may be determined by measuring the thermodynamic equilibrium between folded and unfolded states. For example, Fc variant proteins of the present invention may be unfolded using chemical denaturant, heat, or pH, and this transition may be monitored using methods including but not limited to circular dichroism spectroscopy, fluorescence spectroscopy, absorbance spectroscopy, NMR spectroscopy, calorimetry, and proteolysis. As will be appreciated by those skilled in the art, the kinetic parameters of the folding and unfolding transitions may also be monitored using these and other techniques. The solubility and overall structural integrity of an Fc variant protein may be quantitatively or qualitatively determined using a wide range of methods that are known in the art. Methods which may find use in the present invention for characterizing the biophysical properties of Fc variant proteins include gel electrophoresis, chromatography such as size exclusion chromatography and reversed-phase high performance liquid chromatography, mass spectrometry, ultraviolet absorbance spectroscopy, fluorescence spectroscopy, circular dichroism spectroscopy, isothermal titration calorimetry, differential scanning calorimetry, analytical ultra-centrifugation, dynamic light scattering, proteolysis, and cross-linking, turbidity measurement, filter retardation assays, immunological assays, fluorescent dye binding assays, protein-staining assays, microscopy, and detection of aggregates via ELISA or other binding assay. Structural analysis employing X-ray crystallographic techniques and NMR spectroscopy may also find use. In one embodiment, stability and/or solubility may be measured by determining the amount of protein solution after some defined period of time. In this assay, the protein may or may not be exposed to some extreme condition, for example elevated temperature, low pH, or the presence of denaturant. Because function typically requires a stable, soluble, and/or well-folded/structured protein, the aforementioned functional and binding assays also provide ways to perform such a measurement. For example, a solution comprising an Fc variant could be assayed for its ability to bind target antigen, then exposed to elevated temperature for one or more defined periods of time, then assayed for antigen binding again. Because unfolded and aggregated protein is not expected to be capable of binding antigen, the amount of activity remaining provides a measure of the Fc variant's stability and solubility.

In a preferred embodiment, the library is screened using one or more cell-based or in vivo assays. For such assays, Fc variant proteins, purified or unpurified, are typically added exogenously such that cells are exposed to individual variants or pools of variants belonging to a library. These assays are typically, but not always, based on the function of an antibody or Fc fusion that comprises the Fc variant; that is, the ability of the antibody or Fc fusion to bind a target antigen and mediate some biochemical event, for example effector function, ligand/receptor binding inhibition, apoptosis, and the like. Such assays often involve monitoring the response of cells to antibody or Fc fusion, for example cell survival, cell death, change in cellular morphology, or transcriptional activation such as cellular expression of a natural gene or reporter gene. For example, such assays may measure the ability of Fc variants to elicit ADCC, ADCP, or CDC. For some assays additional cells or components, that is in addition to the target cells, may need to be added, for example serum complement, or effector cells such as peripheral blood monocytes (PBMCs), NK cells, macrophages, and the like. Such additional cells may be from any organism, preferably humans, mice, rat, rabbit, and monkey. Antibodies and Fc fusions may cause apoptosis of certain cell lines expressing the antibody's target antigen, or they may mediate attack on target cells by immune cells which have been added to the assay. Methods for monitoring cell death or viability are known in the art, and include the use of dyes, immunochemical, cytochemical, and radioactive reagents. For example, caspase staining assays may enable apoptosis to be measured, and uptake or release of radioactive substrates or fluorescent dyes such as alamar blue may enable cell growth or activation to be monitored. In a preferred embodiment, the DELFIA® EuTDA-based cytotoxicity assay (Perkin Elmer, Mass.) is used. Alternatively, dead or damaged target cells may be monitoried by measuring the release of one or more natural intracellular proteins, for example lactate dehydrogenase.

Transcriptional activation may also serve as a method for assaying function in cell-based assays. In this case, response may be monitored by assaying for natural genes or proteins which may be upregulated, for example the release of certain interleukins may be measured, or alternatively readout may be via a reporter construct. Cell-based assays may also involve the measure of morphological changes of cells as a response to the presence of an Fc variant. Cell types for such assays may be prokaryotic or eukaryotic, and a variety of cell lines that are known in the art may be employed.

Alternatively, cell-based screens are performed using cells that have been transformed or transfected with nucleic acids encoding the Fc variants. That is, Fc variant proteins are not added exogenously to the cells. For example, in one embodiment, the cell-based screen utilizes cell surface display. A fusion partner can be employed that enables display of Fc variants on the surface of cells (Witrrup, 2001, Curr Opin Biotechnol, 12:395-399, entirely incorporated by reference). Cell surface display methods that may find use in the present invention include but are not limited to display on bacteria (Georgiou et al., 1997, Nat Biotechnol 15:29-34; Georgiou et al., 1993, Trends Biotechnol 11:6-10; Lee et al., 2000, Nat Biotechnol 18:645-648; Jun et al., 1998, Nat Biotechnol 16:576-80, all entirely incorporated by reference), yeast (Boder & Wittrup, 2000, Methods Enzymol 328:430-44; Boder & Wittrup, 1997, Nat Biotechnol 15:553-557, all entirely incorporated by reference), and mammalian cells (Whitehorn et al., 1995, Bio/technology 13:1215-1219, entirely incorporated by reference). In an alternate embodiment, Fc variant proteins are not displayed on the surface of cells, but rather are screened intracellularly or in some other cellular compartment. For example, periplasmic expression and cytometric screening (Chen et al., 2001, Nat Biotechnol 19: 537-542), the protein fragment complementation assay (Johnsson & Varshavsky, 1994, Proc Natl Acad Sci USA 91:10340-10344.; Pelletier et al., 1998, Proc Natl Acad Sci USA 95:12141-12146, all entirely incorporated by reference), and the yeast two hybrid screen (Fields & Song, 1989, Nature 340:245-246, entirely incorporated by reference) may find use in the present invention. Alternatively, if a polypeptide that comprises the Fc variants, for example an antibody or Fc fusion, imparts some selectable growth advantage to a cell, this property may be used to screen or select for Fc variants.

As is known in the art, subsets of screening methods are those that select for favorable members of a library. Said methods are herein referred to as “selection methods”, and these methods find use in the present invention for screening Fc variant libraries. When libraries are screened using a selection method, only those members of a library that are favorable, that is which meet some selection criteria, are propagated, isolated, and/or observed. As will be appreciated, because only the “most fit” variants are observed, such methods enable the screening of libraries that are larger than those screenable by methods that assay the fitness of library members individually. Selection is enabled by any method, technique, or fusion partner that links, covalently or noncovalently, the phenotype of an Fc variant with its genotype, i.e., the function of an Fc variant with the nucleic acid that encodes it. For example the use of phage display as a selection method is enabled by the fusion of library members to the gene IlIl protein. In this way, selection or isolation of variant proteins that meet some criteria, for example binding affinity for an FcgR, also selects for or isolates the nucleic acid that encodes it. Once isolated, the gene or genes encoding Fc variants may then be amplified. This process of isolation and amplification, referred to as panning, may be repeated, allowing favorable Fc variants in the library to be enriched. Nucleic acid sequencing of the attached nucleic acid ultimately allows for gene identification.

A variety of selection methods are known in the art that may find use in the present invention for screening Fc variant libraries. These include but are not limited to phage display (Phage display of peptides and proteins: a laboratory manual, Kay et al., 1996, Academic Press, San Diego, Calif., 1996; Lowman et al., 1991, Biochemistry 30:10832-10838; Smith, 1985, Science 228:1315-1317, incorporate by reference) and its derivatives such as selective phage infection (Malmborg et al., 1997, J Mol Biol 273:544-551, incorporate by reference), selectively infective phage (Krebber et al., 1997, J Mol Biol 268:619-630, entirely incorporated by reference), and delayed infectivity panning (Benhar et al., 2000, J Mol Biol 301:893-904, entirely incorporated by reference), cell surface display (Witrrup, 2001, Curr Opin Biotechnol, 12:395-399, entirely incorporated by reference) such as display on bacteria (Georgiou et al., 1997, Nat Biotechnol 15:29-34; Georgiou et al., 1993, Trends Biotechnol 11:6-10; Lee et al., 2000, Nat Biotechnol 18:645-648; Jun et al., 1998, Nat Biotechnol 16:576-80, all entirely incorporated by reference), yeast (Boder & Wittrup, 2000, Methods Enzymol 328:430-44; Boder & Wittrup, 1997, Nat Biotechnol 15:553-557, all entirely incorporated by reference), and mammalian cells (Whitehorn et al., 1995, Bio/technology 13:1215-1219, entirely incorporated by reference), as well as in vitro display technologies (Amstutz et al., 2001, Curr Opin Biotechnol 12:400-405, entirely incorporated by reference) such as polysome display (Mattheakis et al., 1994, Proc Natl Acad Sci USA 91:9022-9026, entirely incorporated by reference), ribosome display (Hanes et al., 1997, Proc Natl Acad Sci USA 94:4937-4942, entirely incorporated by reference), mRNA display (Roberts & Szostak, 1997, Proc Natl Acad Sci USA 94:12297-12302; Nemoto et al., 1997, FEBS Lett 414:405-408, both entirely incorporated by reference), and ribosome-inactivation display system (Zhou et al., 2002, J Am Chem Soc 124, 538-543, entirely incorporated by reference).

Other selection methods that may find use in the present invention include methods that do not rely on display, such as in vivo methods including but not limited to periplasmic expression and cytometric screening (Chen et al., 2001, Nat Biotechnol 19:537-542, entirely incorporated by reference), the protein fragment complementation assay (Johnsson & Varshavsky, 1994, Proc Natl Acad Sci USA 91:10340-10344; Pelletier et al., 1998, Proc Natl Acad Sci USA 95:12141-12146, all entirely incorporated by reference), and the yeast two hybrid screen (Fields & Song, 1989, Nature 340:245-246) used in selection mode (Visintin et al., 1999, Proc Natl Acad Sci USA 96:11723-11728, all entirely incorporated by reference). In an alternate embodiment, selection is enabled by a fusion partner that binds to a specific sequence on the expression vector, thus linking covalently or noncovalently the fusion partner and associated Fc variant library member with the nucleic acid that encodes them. For example, U.S. Ser. No. 09/642,574; U.S. Ser. No. 10/080,376; U.S. Ser. No. 09/792,630; U.S. Ser. No. 10/023,208; U.S. Ser. No. 09/792,626; U.S. Ser. No. 10/082,671; U.S. Ser. No. 09/953,351; U.S. Ser. No. 10/097,100; U.S. Ser. No. 60/366,658; PCT WO 00/22906; PCT WO 01/49058; PCT WO 02/04852; PCT WO 02/04853; PCT WO 02/08023; PCT WO 01/28702; and PCT WO 02/07466, all entirely incorporated by reference, describe such a fusion partner and technique that may find use in the present invention. In an alternative embodiment, in vivo selection can occur if expression of a polypeptide that comprises the Fc variant, such as an antibody or Fc fusion, imparts some growth, reproduction, or survival advantage to the cell.

A subset of selection methods referred to as “directed evolution” methods are those that include the mating or breading of favorable sequences during selection, sometimes with the incorporation of new mutations. As will be appreciated by those skilled in the art, directed evolution methods can facilitate identification of the most favorable sequences in a library, and can increase the diversity of sequences that are screened. A variety of directed evolution methods are known in the art that may find use in the present invention for screening Fc variant libraries, including but not limited to DNA shuffling (PCT WO 00/42561 A3; PCT WO 01/70947 A3, all entirely incorporated by reference), exon shuffling (U.S. Pat. No. 6,365,377; Kolkman & Stemmer, 2001, Nat Biotechnol 19:423-428, all entirely incorporated by reference), family shuffling (Crameri et al., 1998, Nature 391:288-291; U.S. Pat. No. 6,376,246, all entirely incorporated by reference), RACHITT (Coco et al., 2001, Nat Biotechnol 19:354-359; PCT WO 02/06469, all entirely incorporated by reference), STEP and random priming of in vitro recombination (Zhao et al., 1998, Nat Biotechnol 16:258-261; Shao et al., 1998, Nucleic Acids Res 26:681-683, all entirely incorporated by reference), exonuclease mediated gene assembly (U.S. Pat. No. 6,352,842; U.S. Pat. No. 6,361,974), Gene Site Saturation Mutagenesisä (U.S. Pat. No. 6,358,709, entirely incorporated by reference), Gene Reassembly (U.S. Pat. No. 6,358,709, all entirely incorporated by reference), SCRATCHY (Lutz et al., 2001, Proc Natl Acad Sci USA 98:11248-11253, entirely incorporated by reference), DNA fragmentation methods (Kikuchi et al., Gene 236:159-167, entirely incorporated by reference ), single-stranded stranded DNA shuffling (Kikuchi et al., 2000, Gene 243:133-137, all entirely incorporated by reference), and AMEsystem™ directed evolution protein engineering technology (Applied Molecular Evolution) (U.S. Pat. No. 5,824,514; U.S. Pat. No. 5,817,483; U.S. Pat. No. 5,814,476; U.S. Pat. No. 5,763,192; U.S. Pat. No. 5,723,323, all entirely incorporated by reference).

The biological properties of the antibodies and Fc fusions that comprise the Fc variants of the present invention may be characterized in cell, tissue, and whole organism experiments. As is know in the art, drugs are often tested in animals, including but not limited to mice, rats, rabbits, dogs, cats, pigs, and monkeys, in order to measure a drug's efficacy for treatment against a disease or disease model, or to measure a drug's pharmacokinetics, toxicity, and other properties. Such animals may be identified as disease models. Therapeutics are often tested in mice, including but not limited to nude mice, SCID mice, xenograft mice, and transgenic mice (including knockins and knockouts). For example, an antibody or Fc fusion of the present invention that is intended as an anti-cancer therapeutic may be tested in a mouse cancer model, for example a xenograft mouse. In this method, a tumor or tumor cell line is grafted onto or injected into a mouse, and subsequently the mouse is treated with the therapeutic to determine the ability of the antibody or Fc fusion to reduce or inhibit cancer growth. Such experimentation may provide meaningful data for determination of the potential of said antibody or Fc fusion to be used as a therapeutic. Any organism, preferably mammals, may be used for testing. For example because of their genetic similarity to humans, monkeys can be suitable therapeutic models, and thus may be used to test the efficacy, toxicity, pharmacokinetics, or other property of the antibodies and Fc fusions of the present invention. Tests of the antibodies and Fc fusions of the present invention in humans are ultimately required for approval as drugs, and thus of course these experiments are contemplated. Thus the antibodies and Fc fusions of the present invention may be tested in humans to determine their therapeutic efficacy, toxicity, pharmacokinetics, and/or other clinical properties.

### EXAMPLES

Examples are provided below to illustrate the present invention. These examples are not meant to constrain the present invention to any particular application or theory of operation.

### Example 1

Predictions of point mutations that are favorable in the folded monomer structure can be determined by sequence design predictions using the PDA® technology. A monomeric structure of the IgG1 Fc domain is first created by the deletion of one subunit from a known dimer structure, such as the PDB structure 1DN2 (DeLano et al., 2000, Science 287:1279-1283, entirely incorporated by reference). The monomer structure is then preprocessed by a program, such as REDUCE (Word, et al., 1999, J Mol Biol 285:1735-1747, entirely incorporated by reference), to build protons into the structure. The most preferred placement of protons is chosen based on energetic considerations such as hydrogen bonding, van der Waals and electrostatic forces. The PDA® programs are run to design the point mutations that retain a favorable folded, monomeric structure. The PDA® algorithms use an energy function with terms that include for example, van der Waals forces, electrostatic forces, hydrogen bonding, desolvation interactions, entropy and other terms. Other statistical energy terms include those based on known structures and those that compensate for effects on the unfolded state.

Example output of the design algorithm is a list of favorable amino acids at each site in the protein (FIG. 3). The present invention predicts that the most favorable amino acid substitutions at each position will be those ten with the lowest energy, more preferably those five with the lowest energy, more preferably those three with the lowest energy and most preferably that one with the lowest energy. The mutations should have a low energy in the monomer structure, meaning a better fit for that amino acid at the position. In many positions, the wild-type amino acid is the most favored amino acid. In these cases, the next-lowest energy amino acid may be used or an amino acid from the lowest-energy 4, 6 or 10 amino acids may be used.

Some double mutants in the interface are shown in FIG. 7. These double mutants were designed with PDA® computations that designated two residues at a time may be changed. Although this increases the number of computations to be done, the double variant calculations are important, because the energy of one amino acid at a position depends on the identity of its proximal amino acids. All the double mutants listed have a substantially significant interaction energy between the two sites.

Other double variants, or triple (or higher-order variants) that are important in stabilizing the Fc monomer may be created from simple combinations of single mutants. If, for example, the two sites do not interact energetically, then the change in energy making a double variant will equal the sum of the energies of making the individual single variants.

### Example 2

Mutations that help create a folded monomer may also be designed based on known sequences and structures of monomeric proteins. This approach is complementary to the approach of designing sequences based solely on energetic considerations. Examples of mutations originally designed using comparisons to monomeric Fc homologues include L368R, F405Q, L351S, K392S, T394R, V397E, F405T, Y407T, L368R/F405Q/L351S and L351S/K392S/T394R/V397E/F405T/Y407T. These variants were written using the human IgG 1 amino acids and the EU numbering of Kabat et al. The wild-type amino acid may differ if these variants are put into a different parent protein. These variants were found by first, finding structures similar to the Cγ3 domain structure. This can be done with existing programs known in the field, such as CE (Shindyalov & Bourne, 1998, Protein Eng 11:739-747, entirely incorporated by reference). These new structures are screened manually for those that are monomeric in solution. The Protein Database code for four, monomeric structures with similar domains to the Fc, Cγ3 domain are 1F6A.pdb, 1ZAG.pdb, 1HYR.pdb, and 1B3J.pdb, all entirely incorporated by reference. These structures are examples of viable, monomeric Fc homologues and the incorporation of one or more of their amino acids into the Fc domain are predicted to stabilize the monomeric Fc. The amino acids at interface positions in the parent protein of choice are then changed, either singly or in combinations, to those amino acids in the monomeric structures.

### Example 3

Fc monomers may be created in many isotypes. For example, IgA1 Fc Cα3 domains may be mutated in an analogous manner to the IgG1 isotype Fc Cγ3 domain. For IgA1 Fc, a monomeric structure may be derived from the structure 1OW0.pdb, “one-oooh-double u-zero” (Herr er al. 2003, Nature, 423:614-620, entirely incorporated by reference). The same energy function and optimization parameters can be used as in the IgG1 case. The energies of different amino acids at many sites in the monomer structure of IgA1 Cα3 domain are shown in FIG. 9. To make an IgE Fc monomer, the Cε4 domain must be mutated. A monomeric IgE Fc structure can be derived from the dimeric structure, 1F6A.pdb (Garman et al., 2000, Nature, 406(6793): 259-266, entirely incorporated by reference). The energies of various amino acids at many positions in the IgE Cε4 domain are shown in FIG. 10. The top 10 amino acids (10 lowest in energy) at each position are preferred substitutions whereas those in the top 5 or 3 positions are particularly preferred.

### Example 4

Substitutions of residues to stabilize the monomer can also be found with the sequence and structure approach used in the ACE™ algorithms. ACE™ algorithms use a representative structure and a multiple sequence alignment to judge the compatibility of substitution of one or more amino acids into a position in the proteins structure. The multiple sequence alignment can be created by a variety of methods, included structure based alignment programs such as CE (Shindyalov and Bourne (1998) Protein Engineering 11(9): 739-747, entirely incorporated by reference) or purely sequence-based based comparison methods, such as blast or psi-blast (Altschul, S. F., Gish, W., Miller, W., Myers, E. W. & Lipman, D. J. (1990) J. Mol. Biol. 215:403-410; Altschul, S. F., Madden, T. L., Schaffer, A. A., Zhang, J., Zhang, Z., Miller, W. & Lipman, D. J. (1997) Nucleic Acids Res. 25:3389-3402, all entirely incorporated by reference). For example, the structure of the Fc portion of IgG1, PDB code 1DN2.pdb (DeLano, et al., 2000, Science 287:1279-1283, entirely incorporated by reference) can be used as a template structure and a structure-based, multiple sequence alignment can be built using 1DN2.pdb and the CE program.

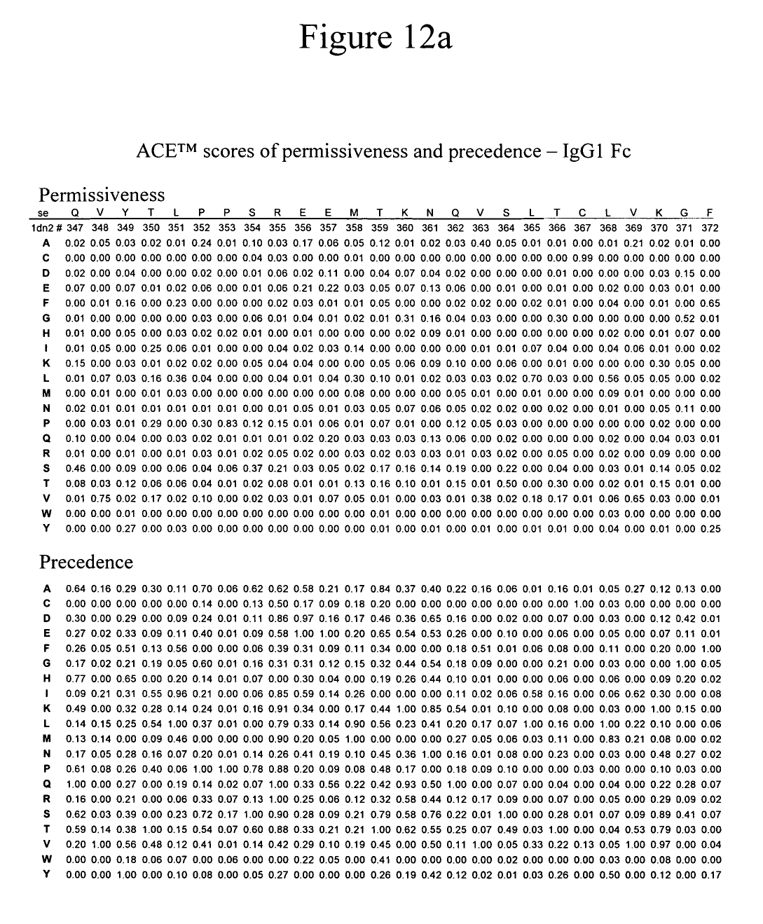

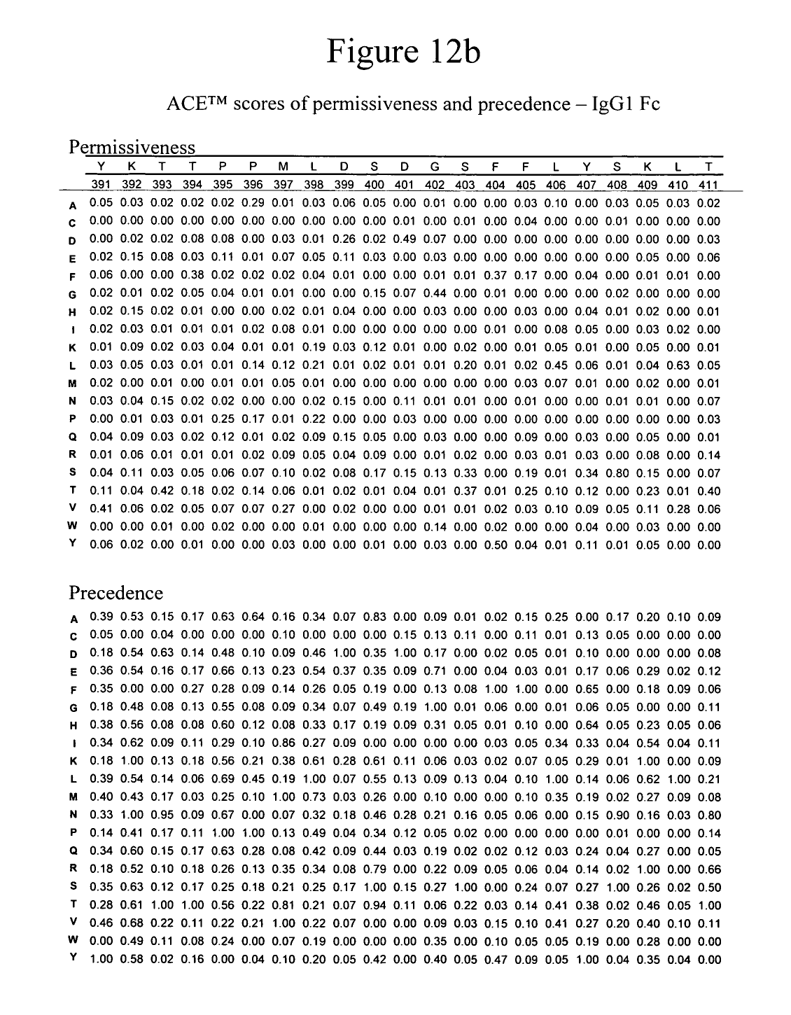

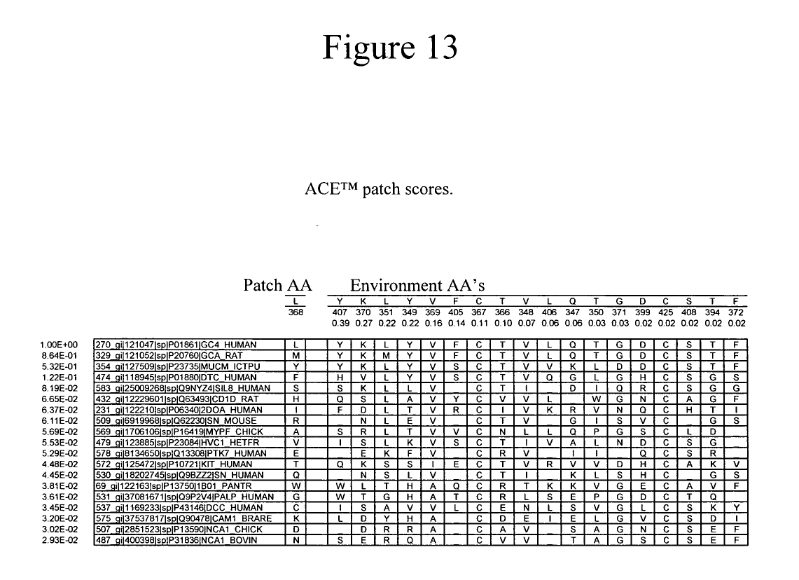

The ACE™ algorithms calculate two scores for each amino acid substitution at each position in the protein of interest. One score, listed in the top half of FIG. 12, lists the permissiveness of the each amino acid into the position in question. (This quantity may be called the “structure-weighted frequency”). This score is based on the compatibility of each amino acid into the environment created at that position averaged over the multiple sequences alignment. The next score measures the precedence of finding that amino acid in another sequence. A high precedence score is given to the substitution if that substitution is seen in another sequence with a very similar environment to the wild-type environment. This score requires only one protein in the multiple sequence alignment to have a similar environment to the query protein. The more similar the best matching environment is to the reference (parent) protein, the higher the precedence score. The permissiveness score, in contrast, is based on all proteins in the multiple sequence alignment. This method is particularly good at finding point mutations that minimally disrupt the native structure, although it is also useful for finding multiple mutations. As shown in FIG. 12, a monomer Fc domain would benefit by substitutions at position 368 to Val (permissiveness score, top panel) or to Met, Tyr or Phe (precedence score, lower panel). As expected, these last three variants are also suggested by the patch scores calculated when position 368 is considered the patch (FIG. 13).

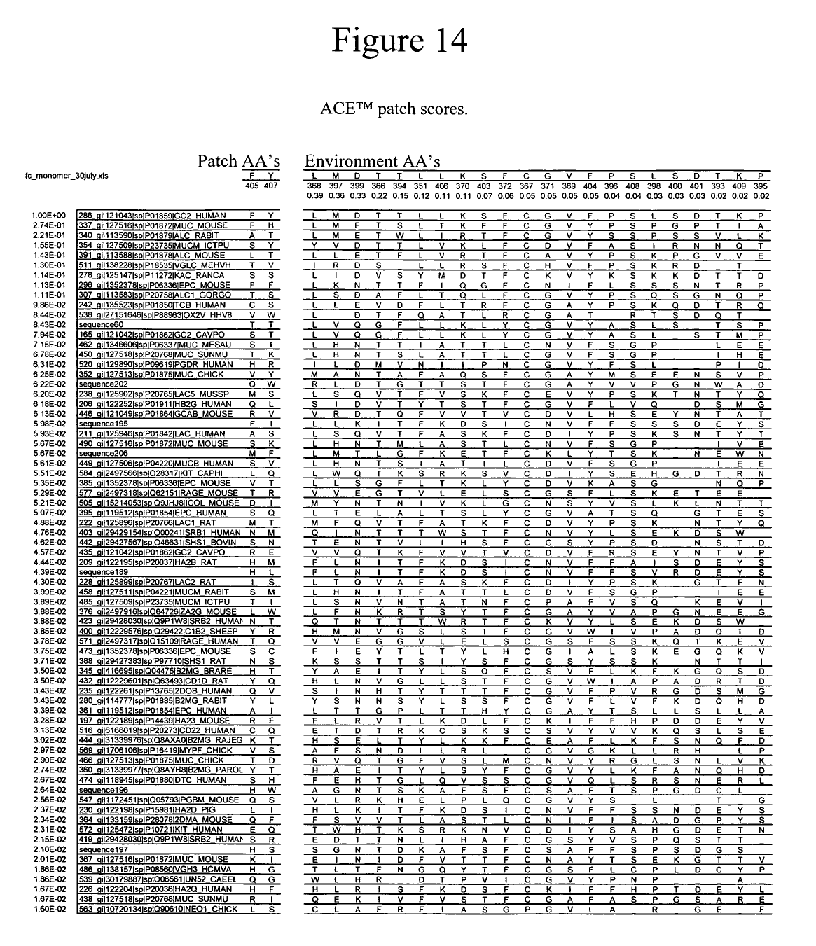

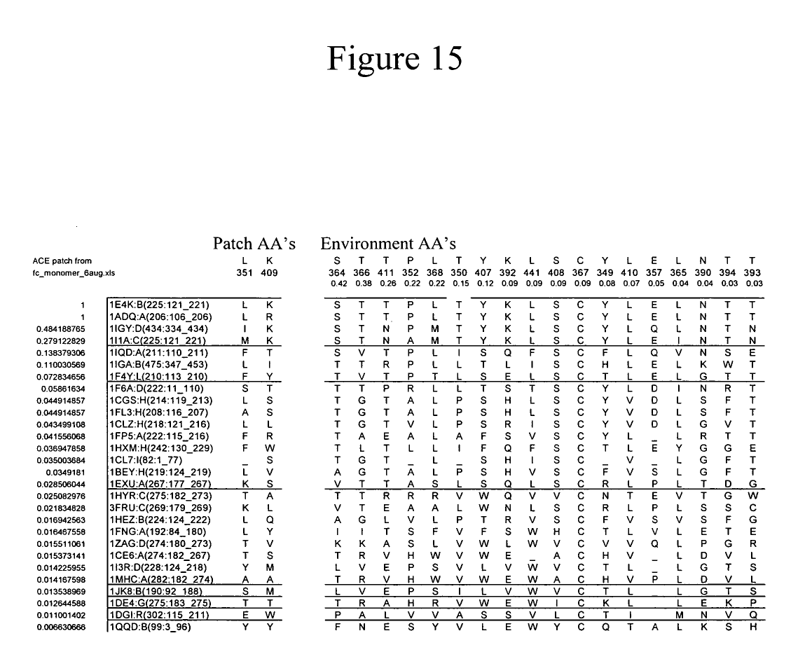

A second ACE™ algorithm judges the compatibility of a patch of residues for a particular environment. A patch is one or more residues that are chosen by the user. Again, the ACE™ algorithm uses a template, protein structure and a multiple sequence alignment comprising the sequence of the template structure. FIG. 13 shows the programs output considering only L368 as the patch. The template structure is a monomeric structure derived from the IgG dimer structure, 1DN2.pdb (DeLano, et al., 2000, Science 287:1279-1283, entirely incorporated by reference). The multiple sequence alignment was derived from the 1DN2 structure using the CE program (Shindyalov and Bourne (1998) Protein Engineering 11(9): 739-747, entirely incorporated by reference) and then expanded by constructing a Hidden Markov Model with the original alignment and HMMER (Sonnhammer et al., 1998, Nucleic Acids Res. 26(1):320-2, entirely incorporated by reference) and gathering sequences that match the model from Swissprot (Junker et al., 1999, Bioinformatics 15:1066-1007, entirely incorporated by reference) . Also shown are the ACE™ patch program output using a patch of residues 405 and 407 (FIG. 14) and using a patch of residues 351and 409 (FIG. 15). As would be expected, the wild-type residues receive high ACE™ precedence score because the exact wild-type sequence is used in the multiple sequence alignment. The second most favorable pair of amino acids at positions 405 and 407 is Phe and His, i.e., a point mutation of Y407H and retention of the wild-type F at position 405. The next most favorable pair of amino acids is Ala and Thr, suggesting the double mutant F405A/Y407T.

All references are herein expressly entirely incorporated by reference. Whereas particular embodiments of the invention have been described above for purposes of illustration, it will be appreciated by those skilled in the art that numerous variations of the details may be made without departing from the invention as described in the appended claims.

1. An Fc variant comprising at least one amino acid modification in the Fc region as compared to a wild type Fc region, wherein said modification is at a position selected from the group consisting of Kabat positions 349, 351, 352, 353, 354, 356, 357, 364, 366, 368, 370, 392, 394, 395, 396, 397, 399, 405, 407 and 409.

2. An Fc variant according to claim 1, wherein at least one of said modifications is not:
(a) a modification to alanine,
(b) T366Y,
(c) T366W,
(d) Y407T,
(e) T366S/L368A/Y407V,
(f) T366S/L368V/Y407A,
(g) L368A/Y407A,
(h) T366S/L368A/Y407A,
(i) T366S/L368G/Y407V,
(j) F366Y/F405A,
(k) T366W/F405W,
(l) F405W/Y407Y,
(m) T394W/Y407T,
(n) T394S/Y407A, or
(o) T366W/T394S.

3. An Fc variant according to claim 1, wherein said variant has an increased content of folded, monomeric polypeptides.

4. An Fc variant according to claim 3, wherein the content of the folded monomeric peptides is measured with reduced disulfide bonds.

5. An Fc variant according to claim 3, wherein said Fc variant is greater than about a 50% monomer.

6. An Fc variant of claim 1 or 3, wherein said Fc variant is a variant of human IgG.

7. An Fc variant according to claim 1, wherein said amino acid modification comprises at least one amino acid modification at a position selected from the group consisting of 352, 353, 395, and 396.

8. An Fc domain variant according to claim 1, wherein said modification is at position 354.

9. An Fc variant according to claim 1, wherein said amino acid modification is a charged amino acid.

10. An Fc variant according to claim 9, wherein said amino acid modification is a naturally occurring charged amino acid.

11. An Fc variant according to claim 10, wherein said variant amino acid is an amino acid selected from the group consisting of arginine, lysine, aspartate, glutamate, and histidine.

12. An Fc variant according to claim 1, wherein said at least one amino acid modification is at a position selected from the group consisting of positions 368, 405, or 407.

13. An Fc variant according to claim 12, wherein at least two amino acid modifications are at positions selected from the group consisting of positions 368, 405, or 407.

14. An Fc variant according to claim 13, wherein said amino acid modifications are at positions selected from the group consisting of positions 368, 405, or 407.

15. An Fc variant according to claim 1, wherein at least one amino acid modification is selected from the group consisting of 349E, 349V, 351H, 351N, 352K, 353S, 354D, 356S, 357Q, 364A, 366E, 368Y, 368E, 370Q, 392E, 394N, 395N, 396T, 397Q, 399 N, 405H, 405R, 407H, 407I, 409T and 409I.

16. An Fc variant comprising a modified Fc domain as compared to a parent Fc domain, wherein the modified Fc domain comprises at least one deletion of at least one amino acid between Kabat positions 354 to 362 and Kabat positions 397 to 404.
